SEQUENCE LISTING (126 sequences)

SEQ ID NO: 1 - DNA, 1500 bp, Yarrowia lipolytica
tgtggctgtgtggctcggcaattggtgggatccttttcaccccagcttcaatggcatctgtcatctgctgctttgggaaggtgcagaacatgctgggtactggagtgtgggaggtaaattgggtccccatagactaagtgtgagacagaggacacgacaagtacacacatcgaatggggactataggcattgatcctaatagcagcagtttcacactagtgtagtggaaaagtagtgtctcaggagtcacgagggcggcgctataacgtagttgtatgtatgggtgcattgagacgggcgtttagtgtgtctttctgtcacttgaaatttccggccctccaatctgggttctgtaagatcgagacaagctttgtgcaggagggtgttgagtgtcactatacgacctgtacgaaaagatacggggtggggacaggtctggagcacattggggcagtagtgtagtgtggggtgcggtggtacggtgggcggttgtggagacgttgagcacggactccaatatggcagtctgggaggctcaattgagtgggtcgtggcgttcacatactcgcgacgattggtctacggtgctccaacaagaccgccgatactgcagacggtgcattacttacccgtgtcgactgagagtctacttggtacttggccctgtggctaagcagtatttgagcaacaatgcaatgcagttgctgactcggttccagatccccttgccccgatgtgtggaagcgttgtttttggggcaagggcatgtgggggctgcatcatactgtggctggggccgttggaagagccgtcggcagcgagcctgagtcgcttctcggggccttattccccccgcctctaggtcagcggcggccgaagtgtcgtactcagctcgcctgtacagtatgacgtgaccgaatagcctctggaaggttggagaagtacagtgcaaaaaaaagttgcaaaatttcattttagcgttcgatccgacgtggcagttggacaatgaatcgatggagacatgatcatgggcagaaatagaaggtctccatgttcaatggcagtaccaattgagcaacagacgggtcgacaggcggcgggcacaccatccgccctccacatggcgcaatcgtcagtgcagcgattcgtactcggattgcatcatgttgcaccgaaagttggggcccgcacgttggagaggcgaggagccagggttagctttggtggggtcctttgttgtcacgtggcatcagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 2 - DNA, 1000 bp, Yarrowia lipolytica
ttgagcacggactccaatatggcagtctgggaggctcaattgagtgggtcgtggcgttcacatactcgcgacgattggtctacggtgctccaacaagaccgccgatactgcagacggtgcattacttacccgtgtcgactgagagtctacttggtacttggccctgtggctaagcagtatttgagcaacaatgcaatgcagttgctgactcggttccagatccccttgccccgatgtgtggaagcgttgtttttggggcaagggcatgtgggggctgcatcatactgtggctggggccgttggaagagccgtcggcagcgagcctgagtcgcttctcggggccttattccccccgcctctaggtcagcggcggccgaagtgtcgtactcagctcgcctgtacagtatgacgtgaccgaatagcctctggaaggttggagaagtacagtgcaaaaaaaagttgcaaaatttcattttagcgttcgatccgacgtggcagttggacaatgaatcgatggagacatgatcatgggcagaaatagaaggtctccatgttcaatggcagtaccaattgagcaacagacgggtcgacaggcggcgggcacaccatccgccctccacatggcgcaatcgtcagtgcagcgattcgtactcggattgcatcatgttgcaccgaaagttggggcccgcacgttggagaggcgaggagccagggttagctttggtggggtcctttgttgtcacgtggcatcagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 3 - DNA, 750 bp, Yarrowia lipolytica
ttttggggcaagggcatgtgggggctgcatcatactgtggctggggccgttggaagagccgtcggcagcgagcctgagtcgcttctcggggccttattccccccgcctctaggtcagcggcggccgaagtgtcgtactcagctcgcctgtacagtatgacgtgaccgaatagcctctggaaggttggagaagtacagtgcaaaaaaaagttgcaaaatttcattttagcgttcgatccgacgtggcagttggacaatgaatcgatggagacatgatcatgggcagaaatagaaggtctccatgttcaatggcagtaccaattgagcaacagacgggtcgacaggcggcgggcacaccatccgccctccacatggcgcaatcgtcagtgcagcgattcgtactcggattgcatcatgttgcaccgaaagttggggcccgcacgttggagaggcgaggagccagggttagctttggtggggtcctttgttgtcacgtggcatcagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 4 - DNA, 500 bp, Yarrowia lipolytica
ggacaatgaatcgatggagacatgatcatgggcagaaatagaaggtctccatgttcaatggcagtaccaattgagcaacagacgggtcgacaggcggcgggcacaccatccgccctccacatggcgcaatcgtcagtgcagcgattcgtactcggattgcatcatgttgcaccgaaagttggggcccgcacgttggagaggcgaggagccagggttagctttggtggggtcctttgttgtcacgtggcatcagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 5 - DNA, 250 bp, Yarrowia lipolytica
cagcgaatggcgtcctccaatcagggccgtcagcgaagtcggcgtgtgatagtgcgtggggagcgaatagagtttctgggggggggcggcccaaaacgtgaaatccgagtacgcatgtagagtgtaaattgggtgtatagtgacattgtttgactctgaccctgagagtaatatataatgtgtacgtgtccccctccgttggtcttctttttttctcctttctcctaaccaacacccaaactaatcaatc

SEQ ID NO: 6 - DNA, 1500 bp, Yarrowia lipolytica
accgacgtggtaccgaggctgatacgatatactgccctggaaacagcttgtactcgtactggtacatgtaacaactgtagcagttaaagcaacaagctaatcactcaaggcatattatagatctatcactcaggacaccttatagatcattctcttcatcagatctatcacttcatcttatagatctaccaaagataacgattcacaaaccacgctctacaattaggaatcctactccgcacccccgcccaacgccaacaggatccgattccattccagaagtggtctttctaaagccggtcgattgtgcggctcagataaagttgatgttcatggtttctttttcatatctgctgattctcagactttgccctggagatcagacatggcaactatgtgtgtatgtatgtacatacgatgataataccaacacagggtaggatgactcttggcgaatcgatggcgagatgttaatcagcagactcggataaggcgaccttttctcctccagtatcatttttctatccgtattcggacttgtacgatactcctgctacctgctagtggttttgatcatgtacattttgatccatgttgatgccctggattcagaaccctaacgccagattacgcacaatggtaccttgtgcttaccccaacatttgctcagccaatgggcgtccgtgtaataaaaacccggtcagcatattactaatcaagtttccacagccttccatggcattccagcgccaatatcgacgcagatacacactctcaagctggttttgccagtcgcccgaccttgccggttagtcttcctcgactcttttaagccgcacaatgtttgcaccgatggttactggcggagatcatgtgatgcatcaggggcaatgtgagagcaagtcccaccagatttatcggcgccgacaagagttttggaccgcggaaacccggaaatttgccctgaccaaagactcaggtcggtgctgcaaccacaaacacccgataatatcgtccatatggctcactccggagacccttgcggtcacaagataaactgcctgttaacgagagtgcagagtcatttccgaaaccttattttggaaatataacggttgcctgattggctgtcggtgggaagcttcgggtgcgtaaagggggggattttgggggcagaaaaccagattagggctgttttgagctgcctagtaatatatggacgttttttccatctgcaaattccgcacaattaaaccagttgcacacgtttccgtggtttatgaccatctcattacgtcaccagttatggggaaccttattgcctctctttctgaactctaaacccagccaatcacagctcgaacatatgtaagcttggtgtcccaacagcagccccggggcggacccgaagttatttaacctccacacattttgatttataagagcaaaggttccctccaacgctactaagtcaacacaacaacactacacacacaacat

SEQ ID NO: 7 - DNA, 1000 bp, Yarrowia lipolytica
ttctcctccagtatcatttttctatccgtattcggacttgtacgatactcctgctacctgctagtggttttgatcatgtacattttgatccatgttgatgccctggattcagaaccctaacgccagattacgcacaatggtaccttgtgcttaccccaacatttgctcagccaatgggcgtccgtgtaataaaaacccggtcagcatattactaatcaagtttccacagccttccatggcattccagcgccaatatcgacgcagatacacactctcaagctggttttgccagtcgcccgaccttgccggttagtcttcctcgactcttttaagccgcacaatgtttgcaccgatggttactggcggagatcatgtgatgcatcaggggcaatgtgagagcaagtcccaccagatttatcggcgccgacaagagttttggaccgcggaaacccggaaatttgccctgaccaaagactcaggtcggtgctgcaaccacaaacacccgataatatcgtccatatggctcactccggagacccttgcggtcacaagataaactgcctgttaacgagagtgcagagtcatttccgaaaccttattttggaaatataacggttgcctgattggctgtcggtgggaagcttcgggtgcgtaaagggggggattttgggggcagaaaaccagattagggctgttttgagctgcctagtaatatatggacgttttttccatctgcaaattccgcacaattaaaccagttgcacacgtttccgtggtttatgaccatctcattacgtcaccagttatggggaaccttattgcctctctttctgaactctaaacccagccaatcacagctcgaacatatgtaagcttggtgtcccaacagcagccccggggcggacccgaagttatttaacctccacacattttgatttataagagcaaaggttccctccaacgctactaagtcaacacaacaacactacacacacaacat

SEQ ID NO: 8 - DNA, 750 bp, Yarrowia lipolytica
caatatcgacgcagatacacactctcaagctggttttgccagtcgcccgaccttgccggttagtcttcctcgactcttttaagccgcacaatgtttgcaccgatggttactggcggagatcatgtgatgcatcaggggcaatgtgagagcaagtcccaccagatttatcggcgccgacaagagttttggaccgcggaaacccggaaatttgccctgaccaaagactcaggtcggtgctgcaaccacaaacacccgataatatcgtccatatggctcactccggagacccttgcggtcacaagataaactgcctgttaacgagagtgcagagtcatttccgaaaccttattttggaaatataacggttgcctgattggctgtcggtgggaagcttcgggtgcgtaaagggggggattttgggggcagaaaaccagattagggctgttttgagctgcctagtaatatatggacgttttttccatctgcaaattccgcacaattaaaccagttgcacacgtttccgtggtttatgaccatctcattacgtcaccagttatggggaaccttattgcctctctttctgaactctaaacccagccaatcacagctcgaacatatgtaagcttggtgtcccaacagcagccccggggcggacccgaagttatttaacctccacacattttgatttataagagcaaaggttccctccaacgctactaagtcaacacaacaacactacacacacaacat

SEQ ID NO: 9 - DNA, 500 bp, Yarrowia lipolytica
acccgataatatcgtccatatggctcactccggagacccttgcggtcacaagataaactgcctgttaacgagagtgcagagtcatttccgaaaccttattttggaaatataacggttgcctgattggctgtcggtgggaagcttcgggtgcgtaaagggggggattttgggggcagaaaaccagattagggctgttttgagctgcctagtaatatatggacgttttttccatctgcaaattccgcacaattaaaccagttgcacacgtttccgtggtttatgaccatctcattacgtcaccagttatggggaaccttattgcctctctttctgaactctaaacccagccaatcacagctcgaacatatgtaagcttggtgtcccaacagcagccccggggcggacccgaagttatttaacctccacacattttgatttataagagcaaaggttccctccaacgctactaagtcaacacaacaacactacacacacaacat

SEQ ID NO: 10 - DNA, 250 bp, Yarrowia lipolytica
taaaccagttgcacacgtttccgtggtttatgaccatctcattacgtcaccagttatggggaaccttattgcctctctttctgaactctaaacccagccaatcacagctcgaacatatgtaagcttggtgtcccaacagcagccccggggcggacccgaagttatttaacctccacacattttgatttataagagcaaaggttccctccaacgctactaagtcaacacaacaacactacacacacaacat

SEQ ID NO: 11 - DNA, 1500 bp, Yarrowia lipolytica
tggactccataacttgacaagagggacattaatctccacctccaggggcttctccggatgttacacggctaagtatcgtgactaacatgggtagtttgctgctcttatgcctgacagaccctacagacatcttttctcaactccattgtggaccggtaggctgttatctgctcactaagctgctcccgactgtgtatatttcttcgctattttaatacttgctacaatagtattaacggttattcattatagcaatcaaaatacttgcaaataccggtacttgtacctggctattcactttcaaaaatcactacaagtatgtccaagtatgtacaagcagcgaactttatttataacacaacggcctggtaaatgaaactctagggtggttgcaaatgtggtggtggttccagtgaaccgagtagtggcggactaaaagtgcgtcacctgcgtgtgagccatttctttttcacaaacagtgattggtcactcccaaaaagagtgttttttctatccaaaagggggccaattgcgcgtgtttcaaagagtggcgcttcagttaccttgcatcgcgatttccttgcggcgtcagagactcatcttcgtgtttgcgacatcgatatgcaagctcctacaaccccagatcttccagtccgagagagccacaaagtttgaagggggaggggtgtgacaagcccaagaagggtgacatttattttccgtggggtgtgtctttgtctacgctgtagttttcgtgatctcattcaatgatttctgtatgataatctgttgggccatcttcagaatgccttgcataatagcaaccaacattgtacctataggtggttgctgcttcatacgagcttgccgtcttatattgagtctaaagggcgaacacactgagaaacaaaaaaaaagagaacggaatccggatctatcagcccacatgcttccagacaaagagtcaaaagacctactgtattaagaccctgaccaatagagtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaac

SEQ ID NO: 12 - DNA, 1000 bp, Yarrowia lipolytica
agtgttttttctatccaaaagggggccaattgcgcgtgtttcaaagagtggcgcttcagttaccttgcatcgcgatttccttgcggcgtcagagactcatcttcgtgtttgcgacatcgatatgcaagctcctacaaccccagatcttccagtccgagagagccacaaagtttgaagggggaggggtgtgacaagcccaagaagggtgacatttattttccgtggggtgtgtctttgtctacgctgtagttttcgtgatctcattcaatgatttctgtatgataatctgttgggccatcttcagaatgccttgcataatagcaaccaacattgtacctataggtggttgctgcttcatacgagcttgccgtcttatattgagtctaaagggcgaacacactgagaaacaaaaaaaaagagaacggaatccggatctatcagcccacatgcttccagacaaagagtcaaaagacctactgtattaagaccctgaccaatagagtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaac

SEQ ID NO: 13 - DNA, 750 bp, Yarrowia lipolytica
tttcgtgatctcattcaatgatttctgtatgataatctgttgggccatcttcagaatgccttgcataatagcaaccaacattgtacctataggtggttgctgcttcatacgagcttgccgtcttatattgagtctaaagggcgaacacactgagaaacaaaaaaaaagagaacggaatccggatctatcagcccacatgcttccagacaaagagtcaaaagacctactgtattaagaccctgaccaatagagtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaac

SEQ ID NO: 14 - DNA, 500 bp, Yarrowia lipolytica
agtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaac

SEQ ID NO: 15 - DNA, 250 bp, Yarrowia lipolytica
cgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaac

SEQ ID NO: 16 - DNA, 1500 bp, Yarrowia lipolytica
gacaacaccattgttaaggggtcggtatctcgctcaatgactcccaccacaagtgtctatatatgaagaggagaaagaggtgcgccagggacaatcccggaaaaactggcaaccagtcatatgcgctgcagtctgcacgttcgactaatttttccggtgggtctatctgaaaagtataagctggaggagatgcacgcagggacggagagatgacgagaggacgaagagggtatttgaaggattaaagacgataaatgggcacaattgacgtagtacacaagactgcgtgctctacttgcacccttcgtctccaattcccctcgtctacacgcatctcttctccccaactgcttgactgcatggtagggtttaatagctcaacagtgagagctggtcagagggcaacaagatcggttgggtttgcagaggtagtgtttagattcaccgacatgtgggggatgaggccaatggctggaacctacgtgtcgcaaacggcgaattgcgcgcttctgttgtttgcaaccagtctccgtggtgtgcagcatacattgttcccgcctctccttgtcttgttggaaggccgatgtcgctgactgtatgtaccgttttttttgtaccgtagtacatgcagggcttggtattttccaactacagtacatacaggtcttagagtgctgattggagatagatatgaatggagtgtacgagtggaaacaaagcgggttagatatgtgtacttgtacatctgtgatattggtagtattgacaagcggtagtcatttcagtgcatcgccgtgccctttctactatccccttgcgccatcaatctcccccttcatcaatccacctctggcagctcttctagaagacctttttacagtctcccaattttatcgtctagtgacggcagaccttgtaagcagatatgtatcatgagtcacgatagctggacagaccaatggcatgcgggcaaataactcccacagacgctctccctccggcgcacaaagcctcgtgctctgaacacgccccagttgatttgacagctctcaacattcgtgtgaacttttttagcgggaaaaagtaacatgacgttgaccgtgcggggctacatgtagcagctgggtgtgctaactacggatacatgcctacaacccccacaagtcaagaccattgcgacgcggaaacaggagcccgcaaaagaggagaaaaacaacggcgagactcgggggcggagtgggtcacgtgactttcctttttcccctcacctggcccgctccgtccatatctctgtcgtacaagacaatattgtcgcaacgcaaaaggtccataaattactgggtagacgcaactctatttgaaggcaacctaccgtttgcttttagtgttttggttttgttaccatatccaaaaaaaaaccatatatccaaaaattccgctgcaccatctcttcttctctccatcaactacccctgcggagaaattcacaccacagttaca

SEQ ID NO: 17 - DNA, 1500 bp, Yarrowia lipolytica
gtttgatgatggcccatgacgagcgaattctcaagctcggccacatttactgtacagccacgagccgaaaggaggaggacgaggagaaggaacaccacacacgactgtccaaattttttacaggccccaagaagaagcgccgaattttcctcgtcaccaccctgtgcagagctttggtggtttcaatggacgacaagcgaatccgtgtggagattgacttgatgtctccttacgtgggaattcgggactaccctgatcgaaagaatggcggtggcgtgtttgccctggaggtccaacagcgagtctactacattgaggacagcaacggatcagcagagtggattcgggccatctccaaggccgccgagtatggcgagttccgagagattgttgctgctcagcacaatgcttcgatcgccaatgctgctgcagcctctgcaatcacccacacgtccacaaccactggtcctggacacgccgccgccgccgccgccgcagctcgatcgaaccccgccacacccaacatcaacaacggcactgctgcgttcggctctcccaccaatgctgcgcagcctggctcgccttacttcgactatgtgggccactctaatgccatgtattcgtccccccagcagcagcagctccccacggcatacggtagcaacacctacaagtttcaggaggctcctgctgtgcccaaggctcctgttaccagcaccacttccaagtttctggctcggaacgaggagcggaagcaactgagacggttgggcaacagacagcctagttagactggtaatcatttaataatatgttgtaatagtgtctatgagtatggcgtagtgtatacaagtatgtactgccggaacaagttcggatgcatcctgagttaattcagcgctgcaatattacgatagtgagtcaactgtcaaaagccgcagcagtgcactaataactaaaaatctggggaagaacggcgcacaagtagaaaatgaagattggccataaacatgaaaagtagaagggcgggtatcagtagggatccatgaagagatgtctgatttgaaacacaacttgtgaaaatgcaaaattaaagtcactgtacggtactgtactacatttgtatgcattttaccactacctagtcttcattcgtacttgtaccgtggcaaggtttggggtcattcatttaggagggcgtcacagtcgtgcactataaagggaaacgaatggcgaaacctgttgcgaaaggtttagtggaacgaaaccagaatttgtaatatagccatattcaattactacaagtagtcttcattggctcctatatctgtatattgtacgacagtacaaccaggctacaggtacagtacggtttttacttcggccttcaaaaaccaaaacacaccaaccaaccctaaccccaaaagatccagaaatttccacgcccacaactataaatgtgcttttctccccacatctccacaaccaaacaaaacgaaa

SEQ ID NO: 18 - DNA, 1500 bp, Yarrowia lipolytica
aaggaaaaccacgtcattgtgtggggtggtgatgtccgggagagtgaggcgtttcaggtagcgtcccagctcaagtgcacctcgttccccttctccgctcttgtggctccttcacctagatcgggctccaacatccgtgagatgattgtgcttcacaagatccagcatcttgtcactgctcctcggtggatccatgccctggagcaaggcatcaacggccaccgaggcaagctcgcgtccctggccatggaccaacaggagcgggacctcacccgacgactccgtcaggagcaggaggaggcctacgagcgatcgctcgcccaggatcgagcccgagaccaacaacgagcccgggaacgggaggcagtggccgaagccgaacgtgcggcagccgaagccgagcgacacaaggagctccaggcccaaaaacgtcagcagtggatcaaatggcgggctggaaagatcaaggagaagaaggaagatgatgcctccacccctactgcccgagtgggtattcgtgtgccttccggtactcggctgaactgcaagttccccgcctattcgactctggaggatctctacgcctatgtggagtgtcacgagctgttgaacggcgatgacgacttttcggacgtggagaaacccgaagactacgaccatgagtactcctttgagctggtttcacccatgccccgaaaggtgatcaagccggtgacggatgtgactgttcaggaggagcctgccatttggcccaacggagcattgaacgtggaggtggttgacgaggaagaggactaagagacagcaacatccaatactttgaagaagctgacaatctagtgtactgtacctgatgtgtctcaaagtcgcaagtacatctttgggtgttcccgtggcaggagctcacggtgctcttcgctaacaggtacatgtgtgtatatgcgcatgcgcgtattgtatcgtaaagtactttcaaatattaaagactgtatatataatgaagtatacggcagttcttgcaaggtgtgtagtggcattcaagcgttgactctaagttgaatctctcaagcattgttgcgggttcaagtttttgtctttggacgtcatcgttgtagaaatggcatcgaacaatcattctaatgtgctttgaggtgtctagaatggcaatatttgagaaaagtcatgatttttgttgtttttttatcgcggaagtttcgattttttacatcatgtataagcccaaaaaactcaaaaaatatcttccgtcccggtggccgagtggttaaggcgttgccctgctaatttgttagctgtcaggcagtgggttttgcccgcgcaggttcgaatcctgtccgtgacgattttttgctggatttcgggattttaagcttccctacttgagggttacccggccgaacacttccaacaatttgcctttaccaatgaaagttgacaatattacacacttgtaaggaacacaaccccacacagtacctgaca

SEQ ID NO: 19 - DNA, 1500 bp, Yarrowia lipolytica
gacaggtgggtcgtcttttgacacaggtgctcaagagtaccggtacatattgagaactggtgtagcatgagagatggatattatagaatcattcgaatcaaaaagagattgtgcattacagtaagttgtctgaactggatgctcccgtccagctacacagcaatgcacccgtgaaatctcagtgaaattatgtaggtgtagcactactatcaacagatagtccttatattttatgagagatcgagcaatctttatgtgtatatatagtgtgggacgtacgagttcttgtactcgttcttctataggtgaaaagatcacattgatgttttatctggaggagttcctgcagcaaattgtaggtgctgagtataaacttgtgacgttaaccaacagaaatctagtcaacattcgtaactacagtatgatctcttatcggctgcagtacatacttgtacgaggagtacatattgtagtacaagtagctactgtgcgctacacctaccaataatcacaactctcctgcgaccgcgtcactgtcctcgatctgctatgagtagaggagaagcatctactattggtcttgttcgtcgcaagtagtatttgcctgtagctaatacatactgtacgtacgtactcactgtgtttttaatagtcggcctcatcttaaatgttcgcttagtattagttttctacgatctggttctgctgctgtaagtgaagtcactgcctgagaggtcgtaccgctctcagttcacgacactggcaccaaggtcacaatccacccgtttaaaaatattcccacaagatattcctatccctctgaaccagttgggttagaaagtagtcatatgaagaagctgaaagtcactctgttgtcagctcgcctcttacaagtagcattgtctcctaacccaacttcatgctccagatgccccaacttatgccccactcgtgtgtggtgatcaggccattggagcaattgcctcaaacagactgaaaatgggtagcaaaagtgagaggaatcaaaacccaccgttttcggactcaagtcataaacaaccatcactccatttccattacactcataacctgtggaccagccatacagaagcgcgacaattccaggtcgaggcaattgggaggacggagaattcaaccaacgggaaacgaaacaagttaaaaacactccaacctcttcaactcggtgatggtgcacggaaattggagaaataaagtcacgtgcccagaaaaatgatccagagaatattattggtaggagggatttcgatagaggatatggaaccacctgtaaagtgctctcacagtggaacgagtggttgttggcggtaaataaactttggggtgttcgccggcagcagccaatcagctgtctctaagtgtagatttgtgtgcgtttaattttcgaagcgtaccacacccagcagtgaaatcgtatcacattactattgtcacaaccacacaacccataccaacacaaca

SEQ ID NO: 20 - DNA, 1500 bp, Yarrowia lipolytica
gttcgtaccagcaccaatgttaggcagaactatacacaagagggcggacgatcacttcggcgttaggcaactgaaggctattttcggctggtactgtaggggacagaggaaacgcaagtgattagtaaatcggataataggcctgttagtttaccgaaatggtgggggaggggttccgtggatatcttgaagttatggaggctgatcgttatttgtggggatggatatcattgtatggacatactgtagctactgtataaacaacggatcttacacctgcctcttgtatgcccattgcttgatcatctatcgtgttactgtacatatacaatagatatagggaagaaaagccggaagtagagaccatagtctggcagaagtaacggcctcgggtcgagagaactataacaaagtccaacggcgggtcttagaatagccccaaggatcacacagttccgcaatccagtttcacatgttccgttgcatggacttttgcatgtctactgttgctacgattcccccattgcaaccacagtttggggttaccccgcattatattagcatgattacgaaagagataagtatcatatggaacatgtgaagggtagtatgcaggtccggcggagaaagagaatgacgttttcattaagcgattcgcttggcggcttgtgggggatgtgacgatacttacggtaaagaccctgtgtgagagctggtactcgctcgttacttcgctgatctgttgggccgtcaatcgaatctcgtggaacttgcattcttcttaactgtgtctatacaagacacctaatgaaacatacaagctaccgaaatcattttactcgtactgaccggtacggtacttgcacaagtagtgaaacttccgaaaatagccagcctcatgcatcatcgcttcaccccttctgttgacctcaaaagcattccaacggtaaaaaattataacgccgccaactggatggttgtgacggcgttgaccaccaatgtgtgggggctggcggtaggaccgagcttattcgtcccaataagctctttggatttgattctttggggtgtgtggtaaaattcacatggggaagaacacggtggcagtttgaggcagaggcccagcgtgtagttcctagggcatgaatataccgaactcatggcgcagaattgagctgaatgcgcaaaaagctacaggatcaaccgcgttagaaatgccgcaaatgtccactaattccccggactgttccaaatgattctgtggggataaatctcaaactgggttaggctttgtcacgtttctttgtgtcgtgtcggttcgtccggggcaatgtgcccacgcttggctgtctccctacacctcggtaaaaactatcacatgctgcccctctcgagcaagcattaaatgcatatagtcaatctaacgacatatatataggtagggtgcatcctccggtttagctccccagaatatctcttattcattacacaaaaacaaca

SEQ ID NO: 21 - DNA, 1500 bp, Yarrowia lipolytica
acaaaaggtagcagaagtatactgtatactcactctttcatgtggaatacaagtagcaccatttatacgatatagtgtgctcgtattcactctgcgaagtacagtacctcggaagtcaatgtttgtggggccacttgacgaaatacgagggtctaatatgggtttttattgtggtttggaaacacaccactcgttccattgtatggatatcttgatgtttcgtttcctctcgaacgaatgcatggtccggataatcttagaagactaaacggagatatgcagcgctattacaaaatgcgcaggatatgggcatctttaatgtgcgttagaggggctgaatgggagagaaagagagagggtgagagaagagtttgccgtttttccagttttccctggccctcgatctcgcttagtcatctgcggagtcagggctgacaaaagtgggttttcgtggatcgctcgtttctgctgtcttctttcccatgccgtggttgggttgggattgggtaatgatgacgttgagtcgtcatgtgacattatgcaaggagacagcgtggtgattcaacaggattaaaaacagtctggttccatggttattttggctcccgagttagagctaaccaccagggcttggttgagcacttgtgtcaccccattgcacggtttgtgttatctccatacgcccgccgggggttcaattgccggcgttgcaccagtggcatgctctgttgtgcttttcgtgactccctatctgtagccatacagtgcacgcgaatgggagtcctccaggcccttgtttgactcccaccggcttcggaataacatggccagtcacaagccagcatgaggtgtagcagtggggctagttagagcaaggttgagtgtagagacctcctccccagccccagctttgtcagccccattgtacaacaatggcataatggtagcgtggcagtatgctgctcctcgacttctctccatgactaatgagtcggtctcggccttccatcgttgttatgcacagatggctttattcctcggcacgtgcgatcatggagggaaagggagtttaagtagcaatgttggtgaacaatagagcaaagagggggaggtagtgtaataaatagggtgtacctgggagcagcaacattcgtggtgcacagggcccgctggaccagaagagctcaattgacagcgtagcattgtccgatctgctacgggaacctgtctctcgctttgaggggtcacgcacagtcattgtctgggcttgcatgtcacatcggactacaagccttttaatttggtgaattttagattttttttgggactgtctgcttttttggttgaggttgttagcccaaatagagttgggcaacatgacacccaaatggcgcacctcagccaagccttatatatatattccccaaccctctgccgcaacacagttgtagtaatccccctcaattgactcaattgaagacacaccaacacacaaacaca

SEQ ID NO: 22 - DNA, 1500 bp, Yarrowia lipolytica
tgacctcatagaaacaaagttgactgacgttgagtgtctcactgagcgacggccacaatacgaggttgagagtggatatctggattgagggtgtcaatatctgttcggcatttgaacggcttgagcactcccccagttttgagagcaacggttcccggctataccgacaatcactcagaagacaagatgaagaggggtgtggagacaagctatcgacatctacatactgtaggaggggaatgtatcatgtgaggcggtttggcgagcgtacaatgtcgtacggtatcgtatgttgggctccattactcaggctacgctaccagtagacccagtacatatgagcatgtctcggtccaaggcctggaacaacagattcttccggcgatcgacattgaagacgagtatggaatttacagtctgattgagtgcatagcttcagagaatggttcttggcccattcagagcgtatagtacaattggtgagatatgatatataccgtatgttatctttccttcaatagctgttgactagattcactatctgtcccgccattcagtctcttcaagtatgcagggtacatccattttttcaccaccagacacgagcgctcactattagctactgtatgcactcttctccgagacattgctgaaacagtggctagtaacgatctaacgagaaaagtgagggagactcgatggtcaaactgcagaagatggacgtgtagctttgtaacgcccctacatctctgagcgaggtgaaagtgctcgtacaacactgtacgtaccaagatgatcttcagccaagtggtggaacatcagacgctgcatcgacagacaactattagtatgtacttcttgtacggtagtaattgggtgagaaaaacaactttctccccttgcaaaaccggctaatgtttccttctttctcctccaaaactcgcacttgtagctaccgtactcgtacaagttccctctctccacaatgttcccatgacattccacgaccttcttttcacttgcttggtagccactggtgcacgcacagcttacatgcagatgctacacaactacaagacaaacaatcgaacgccacatgatccaacggcttgtgcaaagtggatacatggtaaccggctgccgtcaaaagcatgcaatctgtcacatgtcttcccctcgtcttctcctcaaccccaccaacgtctccgaccgctctttgctctcatctctgtctctctcagtgctcgtcactgcacggccctgcataatcgggggaggccaaggtgaaacgctgcaaggtgtgtcgttgggacctacctttggcagtgccaggatgtacttgtgtctgccgccacaagcgtacattcagtcacagctacagagcaacccgacccaaaccacctacaagtagagtactgctgtcaacgtttcttcttacactcaattgacccacaattgacacaaactaccagcctatcaacccaaacacaccaattgaac

SEQ ID NO: 23 - DNA, 1500 bp, Yarrowia lipolytica
atgtgtcattttctaagacatttagcgagctttcataatgtctcaaacaggcaatggctggaaatatttggttggaaaaaatcgtaaaattgaggcgaaatccatctctttctcgtcgagtccttatttctagccattttgtgtttctgggcacgcaggaaattatctgtctcagcgctaagctgaatgccttgacaccttttgctactgaggggaaatgaccggtgagtcggtggtgtaggttcccaacggggggggctgtggaggggttatttggtagtctatcttcgagatggcatttttgttggatgttttctgtaatttccgagtaaaaatgctcatgtaattcccagaaaataccctgatgaccccattgcgtgccaatgcaaggcggatgagcaatggaagatgaacttcgttccaacccttccacgtgacattaaaaatgcagggactccagggtcgttgtcagggagttggcaggggggtctatgtgagacacggaaacgcgttctgacgtcgccatcaagctgctagaagtggctagaagcgttcaaattgttctttgcaatgtgaaagaatgcaacggagatctctttggccggattcgacgcgttttaacggaattggaatctcctaccaaatcgacgcatttttttcacttgaaacaacccgtataaacccacaaataaaggagattcccgggtgagacatttttcatccggtcgagacgagctccacgcacatcgaacacttttttctaccagtagatgtgttgacttcataaatagatagccagatacgaaaatctccgatttctggaccaactctgttgtgtctcagaatgcagcagaaagactttgggcaagtttcaggatgctgcacagaccgcggcataggatttcagagacattctcgtcgaaggcggggcatgacagaggtcgtgttgtgcccaccacatggaccatgcgacatgaccggtgtcatgccgtccccaccacagacacgatgtcacccccaccacagaaccactgttgcgggttacgagggccatgtcacttcccctccaccagaaatcatgtcaatctcggtctcccctcctcttcgtataaccctcgtaggtatgaattataccgttagcgtgttttaaacgtggagaacacgttttgtgctgtatttccacgttctaaaattagtcaaaagcttgaatgggtgtaaacgggccatcgcttttatacggtttcccacggagcgcggtctgagacagaggctcgagttgctagcgtggttgccgattgcttagtaagggtttggcgctgctgagtaagctcgctgagcacgtaagccttctgagtaagctttttttgggtcagctcactgagtagcgttgggagttgcttatataatctgggttagggtacgcacgaaccaatcaaggcacacagaataactttgatatatcccacaccagcatcagcacacacaacatctcaacacaca

SEQ ID NO: 24 - DNA, 1500 bp, Yarrowia lipolytica
cattagctagctagagtccagcttcccggaagtatatactaattcattccttgaatatttatgaaaagcaacatgtgtatttcttgtgtgtgcggcaaacgtagcaattgcaactgcataaacgatgattgtaaaagtatcacactttgctcagacaggttagattcacctggtacgagggcagtgtcttaaaggttccatctacctcggcccttgtttcttgaagagtggtcaatatgtgttttatacagctgaaatttcccctgtatgttgagatcgtgtatattggtcataatctgggctctttagtcgatcccagttttctcgggcaagtttttttctccacaaagtaccgctggaaaactctatgtgacttgttgacagattacttgggttatctgcgggatatgtcttggataggcaaccgggcatatatcaccgggcggactgttggttctgtacgtacatacagcactttgagctcatgtctcacacgcaaccatggtgcgtggaggctttggcatcctttctacttgtagtggctatagtacttgcagtccaagcaaacatgagtatgtgcttgtatgtactgaaacccgtctacggtaatattttagagtgtggaactatgggatgagtgctcattcgatactatgttgtcacccgatttgccgtttgcgaggtaagacacattcggtggttcaggcggctacttgtatgtagcatccacgttcatgttttgtggatcagattaatggtatggatatgcacggggcgtttccccggtaacgtgtaggcagtccagtgcaacccagacagctgagctctctatagccgtgcgtgtgcggtcatatcacgctacacttagctacagaataaagctcggtagcgccaacagcgttgacaaatagctcaagggcgtggagcacagggtttaggaggttttaatgggcgagaaggcgcgtagatgtagtcttcctcggtcccatcggtaatcacgtgtgtgccgatttgcaagacgaaaagccacgagaataaaccgggagaggggatggaagtccccgaacagcaaccagcccttgccctcgtggacataacctttcacttgccagaactctaagcgtcaccacggtatacaagcgcacgtagaagattgtggaagtcgtgttggagactgttgatttgggcggtggaggggggtatttgagagcaagtttgagatttgtgccattgagggggaggttattgtggccatgcagtcggatttgccgtcacgggaccgcaacatgcttttcattgcagtccttcaactatccatctcacctcccccaatggcttttaactttcgaatgacgaaagcacccccctttgtacagatgactatttgggaccaatccaatagcgcaattgggtttgcatcatgtataaaaggagcaatcccccactagttataaagtcacaagtatctcagtatacccgtctaaccacacatttatcacc

SEQ ID NO: 25 - DNA, 1500 bp, Yarrowia lipolytica
agatggtgctgccaggagagagatatgactaaatggtctgtctggtgagcagctcaggtggcccaaacgtctcacgtaagcagaacctactactggttcagccgtgagacatcagatgacgtttaccaacgaacgccgttgaaaccgtgctttgtgtatatcatgtgtgctctataataggatttcggttggagcgataacggagtttggacaccagagttcgcgcattgcacgatacaccaacgttttcacattatcatgtaaaaagtgtacatacatactttcacaagatggtccaagtacgatgtagtgttgtgcgacgggtgtgtctgtgtaacatgtttgtccaaatatatataggtcgtcagtagcccaagtttctgtttcccgactctatttcctgtctggtaggctcctcacctccccgtcacctcctcaccacctcctgacgtcatgatcgagacgcttatcgccagtaaagccgaaccgaaccgaatccccacaataccccacagtgtgcatatcaaacctaccggttgttgctctctccagccttactaagaaggaggcgacgtggcagtggctcgcgggaggatcggcgggaaactccgggatatccgtcgagagtttacacgtgaatgggcagcgcaatccgttgacgacgatacgactggcaaagtagcgacgatacctgccagacaggtgacatgtgcaggccgcactaacaaggaaacgggcgctggggggggcgggcttctagactttgcccttgaacaggaatctagtgggggcttgtctttccgccaatgggggagcgcctgttgagcgaccgtgcatgctggaacgccaagtgtatgtacagctggtggtctcgcagcggtatgtgacgggacttacatctctcgttttttcatgaccacgttttcacaggctcggaggtacgttaaagttttgaaggctgcatctgaaccgaggtatgggggagtttgaagagcaacagtgttggggctgagggggccaagatcggggcaagcagaggtcttagatcaattgtggggatcccaaagggctcgttatcacctttttccacccaattcgggtcccaattgatccactactggcttgcccaagttaccccagaaatgccgccccggatttctccaaaaacctaataagcttcatggaacttggtggaagtgactttctacagagtggagagaaccgtggacacgtggcaatggcgctgaccgtgtccccgagccgaatcgacgtgaggggagaacggagtatctgcggtcatgtgaccttccagagcggcgtcgccagtgtgcacgcggtgacccccagtttgggtctctgtcacacgcatactacctcggctctccacatgctgaactttatctttcgtggggatcataccgaaagttgcaactaccaggtgtatataaagcctggtagactccccccactttggacctcatccaaccaagacacacaaaa

SEQ ID NO: 26 - DNA, 1500 bp, Yarrowia lipolytica
tttcggcgtgcaaaatcgcacgaacgtggtccgttatggaactcggcgcaccacgcgttcatctggagcccgtaaatcagtgtcgagaacgactcgttgaagttattgaggttggtatggatcacctggaggtccttgagattgcggtctagcgctgacatgccgtctgcgagttcggccatctgttcgccgatagccatgtccaacacggactccactatcggatagtgctcttcgtggtccgattgtcgcgaatttgagcgtcttaatggtgttgtgggtcgttgcatggtgtgagtagattaagttacgcgttggtgatatttataattatcgaagtgtataacctgagctgcagtagaatcgagtatacacaggttatgagtatggataatgatggaagtgtttgataatggtggtaaaaaacgactgtggggatttggagccgatctgaagacttttgggagcgacagtccgaatctcatttgacgacatatttattagttggatttagttagaaattagttgactggaaaagtcacctgggggttcatttctggtgttacaagaatggaagaacattgagatgtagtttagtagatggagaagacttgagttctaaacaaaagagctgaaatcatatccttcagtagtagtatagtcctgttatcacagcatcaattacccccgtccaagtaagttgattgggatttttgtttacagatacagtaatatacttgactatttctttacaggtgactcagaaagtgcatgttggaaatgagccacagaccaagacaagatatgacaaaattgcactattcgatgcagaattcgacggtgtttccattggtgttatgacattcatctgcattcatacaaaaaagtcttggtagtggtacttttgcgttattacctccgatatctacgcaccccccaacccccctgctacagtaaagagtgtgagtctactgtacatgcttactaaaccacctactgtacagcgaaacccctcagcaaaatcacacaatcagctcattacaacacacccaatgacctcaccacaaattctatacgccttttgacgccattattacagtagcttgcaacgccgttgtcttaggttccatttttagtgctctattacctcacttaacccgtataggcagatcaggccatggcactaagtgtagagctagaggttgatatcgccacgagtgctccatcagggctagggtggggttagaaatacagtccgtgcgcactcaaaaggcgtccgggttagggcatccgataatatcgcctggactcggcgccatattctcgacttctgggcgcgttgtattcatctcctccgcttcccaacacttccacccgtttctccatcccaaccaatagaatagggtaaccttattcgggacactttcgtcatacatagtcagatatacaagcaatgtcactctccttcgtactcgtacatacaacacaactacattcaaa

SEQ ID NO: 27 - DNA, 1500 bp, Yarrowia lipolytica
catttatcgacccatcgaccctccccgacatgtcgacatctcccggaacccctattcaacacgtccctcaaacaacaacactcacactcacactctatcgccattacaccgtgagggctgtgctgtccaaatagtacacccacagtaaaagaacaagttcagccacatttttgtgattgtgtgacattggtttcaaaaacacaccgccaaacaactacagtttgcggtcacgtgaccaattgccatggtccacgtgatcccgatcccacgctccgcacaatctacaacccccttcccctttaccaacgccaccgatcaccgcctttcagttgctctaacttagaaggcacgtggttgcgtccgcaggacaacttttccggtcagttactgggcttgtccctcttctcttcctgtgggcgcctccgacgggacgctgtttagcaaccaaccaatccgtcccagcagagattgctccagatctttacataatccacccgagtgacgtcatgcatggcctgattatcttgccgaggtacggcctcgttcaggctatacggaaaagttttcttttgacgtttttttgagtggatttccacgagccatttaggtcatggttggatatcaggtcatggccttggtacgagaagcatcttgtcgactgattagcttggactcatgttatctgcctcggtagcaactccagtgcgaacaagcacactgtactgctgctcacatgcggtttcaaatgcacggggagacgcccagtgccaatgtcgccacatttccaggtcgtcgagttgaccttttccgcacaattgagtccacattgtctacttggtcctggtactacactcgtaccggtaccttttgatccgatggttactttttatgttctattttacattagcgtggaaatagaccatgccatctttggcaccccggaaaaacttgatccaatagagttgttgggtggagctagtgactggcggcaattggagagcttctagaagacgaaccaggagcccgataggaactccgttggcgtgagtcggccccccagtagcaattcgaatcacgtgacgtggagttttccctcgcccgcgttcctggattgtcccggtgtgacgaggccgactggatttgatcacccaaccccacacgacgcataatgtaaatgtatcatcatacagtacatgcccgagtctaatgattggctggttcacggggaccgggagcgtccagtgggccgtatggcggtggttcaaacacctcagatcagctcactatcggctgagacaatccttaactgttggtcggttgccgtttttgcctgctcctaacagctcgcaccgactctaaaaaacctatacgacctggccggcgtaactttgagtgtcgtcaaactctgatatatatatagagagacgtatcccaacagttgatagtcgacaaacgcaaaacagacggacactgaaccccccgcgcttcaaaacaccgaca

SEQ ID NO: 28 - DNA, 1500 bp, Yarrowia lipolytica
aagctccaccgacacacggaaagcgcgcgcatagatgttactcatgccatggtacagcatggtacacgatctgacgtgaaacctacgccactaccagtgccaatatagcgccggtagtaattcctttaaacctcataatcgccgatggtcagtgttagcggggcgcgcggcctaattaccagtcgtactgcgtagcttaacattcccattcgatgctcccaaccacacggacatgccaggaggtgttctcgccgtgttccttttgttaggggatgtgcgtgtgtgtaggggtacagctcacatattgatttggctggaaacgggcgacaatattatttcgagactcaggacagatagaagaaacaaatgaatggctataaccggaacagaccgtatggttgcggccagacaaggaggagagtaccttggtgcgagtacttgtagtgcacgggcagatgccgacgacgttaagaaatacacattccagacagggtccagggcctacggggtttaagagccagttctggcgattgaggagaccacttcgagcaagttgcctggagctcagctccggctgcctccccctttgcgcctacattctccctcattccaaacaaggttttttacacccttgtccgagcccatgtctgcactcaaaatggctgcgccagaacaaacaagtttgagtttccaaaaccacaaaagcaaagacgccaaacgcacccctatcctatcccttttcaactttgtgtgggcagagaggccaaacatacaaaagtgcatttgggcccaaagcaagaaaattgttaactcatccacttcttttcgacacgacgttctaacacattttatcccgcacctggtttgtgcgtggagggtgagtatctacaagatggggacgctctgtgccacacaggcagctcctacaaaccgaggctttcatttcacacggattttggagggttttggggttttggaggcacaaacgacaagaacaccacgacgagaaaacacgagcgacaatgctggagactcagagaagggagcatagcatagcaggccaatcgacagcagcgggccgccactcgataatgcgttaatctaccgtcgacactggatatgttcattgttattttcctcttcctacacgcacgtgtgttttttttctcccgacccatacaccgtacatcctgttgacccacacacacaaataaggataagctctggaatgtgtgggatgtgataccaagacacggcgacacgaccaatgagataaaaatgagaccatgagaaatgtgttaaccacgatattattgtgatattactggagtgtgtgtgcctgcctttcccgcaccatctccaccgtcattccacgctctactttgtttgtgccactctaagcccctgccatcatccatgccctccatcctgcaacgacacatactaaccacagtccactcattcactcgtttgcatattattttacccagttaaatcgtt

SEQ ID NO: 29 - DNA, 1500 bp, Yarrowia lipolytica
actccttccagaaaaatgtgatggcggcccaagggttggacttgacgtcgcgcgactttttgctggtgccccagttggagtagatgatgaggccacgtggatcgagctccttgaagagcacagttcgcgagctgacccgaccggacggcagctgcgcggtggcgaagttgactgcctcgggaatggggatgccggcgtccttggcctgctggaaccatttctggaactgcacaatggggtctggactcacgtcctgcacgtccagagtcgcctgtttgtactggtgcgtggggtttgctgtgatgattttgtcgaggttcattctggaggtggagaagcgacggagcgacggcagaatgcgtgacatgaagacagtggcagtcgatgggaggtttgtggggtgatgtgttgggaggtgcagcaaggtacgatagtttttacatcggggcttgggggagtttatatagacggcgaaggtggctgtttgtggtcgacagcggggaatttgggagcaaaagggagcgggaacaatgtctgctgcacagtggaaaccgattttgggacgcgaataacccagcttgtgactcacagaggtgtatacaagtcaagtcacagggctgacttgtgggtggcatgatttatgtgcactacacgattcctggagtctaaatagccgcctggaaggggagagatattggagtaactagtaagtgaaatgggtcgaaggtcgagggaatcaaatcaactgatgaattgttgctatttggtcaaaaaaatggacgatttgggcaaaaattgaggggtttttgagactctgaaaactgtcaaaatattcttcgagtcattttgagtttgactaatatgatgatgaaaaaaaagtgattgttgttggacgataatagtgattagtaatggtatacagagtgaatagacattctccgaggtactgcacgtactcatcagagaacccaaaactcttctctgccatcatttgcaaaatccgcgaatcgaacttacacatgtccatcttacatgattatgtaaccaaccaccatctgcgaccggatagaccccctgtccatgacctctccaaggtatttcgctcacaaaaaccgctcattccgtctatatcctcgtatcttcatatcttaatatcttcgtatcttcatatcctaatatcctcatatcatccatatcgtctatatccccaatcatctgagctgtacactcttcaatctcctcgcctccgtaactcagtttcagtacaactacagaacatcctttcagagtcattcgccagttggttttggagctgcttgtctgacaattccagtggatggccgcattcgtctcaggaaaatcatcaccaaaaatcaccccaattccccctgtcaatcccgacccacagacctccccgaacccccctcataacctttaggattcacatggttagtcccaaaatgtgggtctcttctctcaaccaccaccaccaccaccaca

SEQ ID NO: 30 - DNA, 1500 bp, Yarrowia lipolytica
caccctcgtcgtcgaccatctctcgtgagaaatcaccattaatttctgggaaattcttttcgatttcaccctctcaatttccctctggaacttggcaacgggtgtgtacataatcgaggccccgtctagagttagagttccgaggcggtttgccttggactattactgggaactaggcccttgacgataacgcggaatggagtagtttgagggcaaacaggacatttaacgggggtgttttcgatgagaaaggcgatgaccacatttccccagtggttgattatacccatactccgttttttgttttatcgccaatttgtcagagtaacccacatgtattgtttttttagaaaaaaacgtctaaaatcagctaaaatagaaagatgagtgctaacacaatacgcgcaattagaaactgctcaatggcaccgaaaacaggtgggggtttgataactgctcaatggatccaagattgacaatggatcgaagtagtcaatggatcagactgtcgattgaagagaagagctcaatgatactgctgtacgcgtgccaaaccagtcaacccacgtcagatgtgacgaaaaacaggtattaccttcaacaaacagattctccgtatcccgggactaactaaccttctctcctcccaccttgtctcgatccactctcgtctaatgtcacataaatgggcccatgtggattatatttggtatatccgaaattaactttttgttcggaaattttcggagttggcacacatgtctcgtgtaggttatcaccgacaaggccgaagccgaggtgcgcactctctcacaccacacaaacgcgtctttaacaggcgaaaaacgacactgtcgacacaatcatacgatgacacaaaaaatggcagaaaaaaaagacagtcaaaaaaatgacaggctgagacatgagagacaagaggagggtaagttagatcgagagttgacaccaaagatctctagtggtcgacgacaacccaccaaccaaccaaataaccaaataactcccccgttggacacaaggatccattcacccgcgaccccgcacgaggcgttgttgcagcacatacacacacacacagtgggtggttctacagtaggtgtatacactctgaggttggcgcttgagggtaacttttttttgggactttgagtcaactttgatggataagtccgacgaaaggagttgggcttgactggccaattgtgggcgcaatcggcccacggaaaacggaattctgtagtgggcagtcaattgaggtgacagagagcaaaagctgtcgcaaaaggcgcccccaacacctacctttatgctcatggtctgcctacgctctcccctcccctcaagtctcttcacatgcgaaactttaatctaaataattactcaccacgtattacactatttgtgcagcgcatatctacattgtgcctgcggtagaccaacctcttctcctcctcacaactccaccagactctcaaaacg

SEQ ID NO: 31 - DNA, 1500 bp, Yarrowia lipolytica
ttcggggttagtgtacgtaccgagggtgttttgtaattgatgagggaaggatatatttgctatttcgacatgagaaaacgattcgaagttggcacattagtcatctggctatttttcagctcccaaatcggcctacaaccccctttcttaacacctcccaatgcctggtggcacctctcatacatctcccaatctgtagagacaaaaatacacttcccattttaatccgcatttttagcacatccagaacaccgtcttctacgttctactgtacaccacagaaattgcatagtgagtatcgagaggtttggctaggagagctgaggaggacgagcggaggacgataggggcgtcacatgacgacgattggatatgctaccacagcgggtccatgccacaaaggttcaaacgccggttgtgtgataggtggcgagctgactagagcactgtagtggacagagatggagacagagatagagacagagacggacctgaggggaagacaaggcaattctgcgttacagaggatgtcctctgtccgagtctgttgtggtgatgtcgctgctctctgttcgtgtttgtcgtttgtgtttttggcagttgttggctggtttgtgggaaaaggggaaaaggtgatggagactatctcagcagatagcggtgattgtgtttatgctgtcattagacacaatcgtcgatgggcttgttgctgagctctctactgacgttgtcgttctttgcgtgtcgcaaactgagcgataacactgtttaggggtcacagcggggatgagaactacggaactgttgcgcgtgcacattacgagtgaagtactcctattttactaatggcacacacacttgcaagacttacttgcagtgttactactgcacttgtgttgtacgcccatagacgccatctgaaacatgtctcaagcaacgaactacgacacagccatcgcacccagctctatcaacagaaccacttgaatggtacaagcgagacagtcaacccttgacttgctgcgacttatgacacagtggtttgtcatccatgacaagctcttctctgcgatatggttcgtgtcgtgggtacaagtgtgcactctgcagtttgctactttctactcagttatcgataccactttgattgtcttccacttaaccaccgcttgtccctcgatctatctcgaccgagtctcacaagctaaatttcaatctgagacatgcagatcgtcagtgtggagagctttcttgtgtgatcactgtagccactgaggacaccagtactacccaagactttccaaccatcgtcattaccactcccgtcaagcaggcacttatcaggctcacctgccaagccttattagctaatgtttctttcttttcgctccttagcttgtgcctctgcgagtacataggtggatcccgcatcttcgctcgctctcctggcctctgtcccacgtattcgtaggacacgcacatcaaaccgaactaacacag

SEQ ID NO: 32 - DNA, 1500 bp, Yarrowia lipolytica
tgtatgcagagtgcacccaattaggcagctatattgctttggagtgcgtcgacatgccatctctcattttgagtttcgagcgtaaatgttaacacaaaaacttaaatagtgaaagttaaaatgatgagaacgacgtggtcagcatcgacgaggcggagccgtgtaggagaccaccgtggacgaatactccacgtcacgtcacgtcacgtcttgcgcaagctaatgggctaatgggcgacgttattagcgggtcttggtccttgtagggagtttgggaggtcgcgaaactgtgtgtatttgccaaaatcgaccacgtgcacgtgttaccgtacacactgtacagtacgaggctggttgatcggcagattctgtattgtaccggtagttctacagcatgccgacagagcgactggggagctgtgtgcgttctgtcggtggttacggtggtttccaatgacccgatttctgcgtgcgaaaacgaagtcctcgtcgttagatgggaaaaacggaaaaaaagaaaaaaaaaagaaaaagaaaaatggaaaaaagaaacgtaaatcaaaaacacccaaatatgtcgtcgacagtacgatagagagatagcatgaggaagggcggcggatggaagctgtttaatagtcggcagtatttttgaaattattcataataccacgaggactcatgaattataattattcgagttgaattttttcctatatattcttgcattttacaaagatttgtttttttccagcattttattttttataaatcgattttattttattttattttattatcctcaccatgctccagaacagcttcgcgcatcacttgctgtacaagtcttatatacagcgtgtctgtacagtgcacatacatacacattaatgcaatgcaatttacatgcaggataaagttcaaatgttgggcggctgtggtgaataggaaatagagaataggggtgcgaggagagggtgatagcaatagtgatggatggaaaaatggaaaaatggaaaagaagcgtgagtgaaaaataggggttttcggggcccgattgcaggtatttttcaacaattacgcggcctggcatcccctgaaccctcgcccagtgttttttgccgccaaaccggcccccagagccccttctggaccgcactttccccttcctaacaccaccaccacacatccacatcacacacacggcaaaccaatcttgccatattgacttgtttttacctttttaaactttggaaccacgtccaatatccatgtagccctttgtttgggctttgtttgtacagtgctggacagggtttcattgttctcttggctggctagacgccgggtcacccgcatgtgtgcacaaccctccaaactcaatctctcacttatatatacaagaccaccgtcgccagccaaacccctttctcttctcttttttccttcttctttctagataaggtccgacctaaaaacacatttcgaacacacaacaca

SEQ ID NO: 33 - DNA, 1500 bp, Yarrowia lipolytica
gcaggtctgtgattggttgaaaactgggattagacgtgacgtacgtgtagaaaattttaaccgaaagattggaacaaagaatatgtaacggacaaagaaagactatgcatatgataatgtcaaatagagtcacagacccaacttagaaacccgtgggtcctgtactgtctctcattggacgaaatagggttggctatcgatgtaggagacaagggacactggcagctggagcgcatattaccagcacgactaagtaaaccgtttcaccaccatggtgggggcacgccgtcagtatatcgatagtgagaaattgtaggttgaacactagttaccacatgtttttaacgcttagttgggccactctaaccctggggcccataagccttagtatggagcaagatcgaggcaaaagtccatcagagtccacactagagcggtccttaacgtaataccctgaaaccatcaccggtttcttgcacacgaccatgtatatggtataggtgcttcaatcgcggcctgggaattggttttctgggggtgccaaggcatagatgttcgggagccggactactcggacgtcatcagccccccgacatcaatatagcgcgaatagatacggagtacaaagacgtagtgccaatgacaggcagaagtttattgtgcgacacacgtggctagcgaccttgactattacttgacggtggtgaaaagaagcggaggccatcgatttgaaggcttggcgggtcttttgatccttctaaacgatcccaagacaatgaaccaacgttgccgagctcttctcaggcctgacttggcgctgtagggtccttaaattgtcgtattggcacttgcctataccactgaacctctcctacaagacagatagcctgcaaatatcagctgtctgccagagttttgtccccacagtcaggcagtcagttaagccggtggtggcctcgtctaccgtgtgtgatcaaacttaatggtggagggggtggtgctatagtaagtggctctcccagggttagaagggccaagcgcaaacaagtagataagacaggcacgctagcgggaagatccactcacattgccaagaagcgcgaatatcggggccaagcattgcgatattaaactggagatagccactgaaatggtcgatatgggactcgtcttttccaacttctcccaaactacttgggagacgcacggttgggttgagagtgcctgtgtagtgtagtgctgagagagctatggcccagctaactatggttttgtgttttaccactttttgaaaatttaggtgttccagaactgtattggggaccaagaataattagagctaacccaaaacggtaaaaaattaaacagagttttggtactcatcggaacagaggaaaaagggaaaaaaagaccacctgtgcacgctccagtgcatatataaaccacctgcgcttcccagctcaattcctcatcgctatcgaccccactctcaggcattca

SEQ ID NO: 34 - DNA, 1500 bp, Yarrowia lipolytica
tgcgctggtctcatccgatggttgctctcaaagctcccagacctggcagcaacgtgggctcatagcaacatgagtcctccatgctgctgacgcgaaaaaaacaatatacacctacaataggcacgccgcgaaaatctgcaccccccgatctccacaacccggcaaaaaaaccctatcagccggcaaagaagccaaagaagtccaaaagagccaaaagagccaaaagagcggagacaacagacgccaaggttcggacggcgtggatctgagggtttgctttagcaactggtttagccgccgtgcgggaatgcgttggtatccagctaagtatgttcagctctgtaagaacaaggctagacagacacctcgaaaacgtcggaccattgccgccgtacactaatcccgtgtcgccgctactccaatacactgtgactctcctatgtccctcgcgtttaaacaaagtcatagcccacggcagttaccggagccctaggttctgcagtgtgtatggttattttggaaaaaggaaaagtgggccaaaccaaaaccagacgtggaccttagttatcgtaggcttgattcacgagtaaagggctcgcgctatccgtcggtgaggctcgacaatttgacaacagaatcaaaccaaaactggaaaccaaaactggaaacgaaaagcagaaaaggaagtgaacgagtatgtacgagtactcgtactggtaagaagccttagacacaactgcgaataccacgtgtccgtgtacctgccgtatacctacagcgttctggtccccaacatcacgcgataagtgtacaagtaccgtacgtactcacacctatgtgatcactccatgctcactctgtactcactccgaccgatatcggtaacatcacgtgtttccgtacggtttggttgtcccatgccatgcataatatcccattgactctctcgctaaactgcccaaactccgtgtttttccttttccccccacgcacacgcatgcaccaacgtttttggatcccagtttagatgccgtcgttagttcctgtagttgcctgtgcaaccgtaggtttgatggggctgattggattggaagtaataatataatgactcagtggcgatgttacgtggtggcaaaacagcgctattttgcacatgtctgcgaaggactggaccaacggcggggtccttggagcccggccatgctggtgctgtagttgctaccttccgtctggctcatagcttgcctgcattcactgttgtccgatcgactatgaatactgtacgattcttactgtaacgtgcaaataaacttcccaggtcggacatgcttccacagcaaaagctcggtgaacaaggcctgtcggagatttcggagtaacgtctgtctgcaacttgttcttgttcttctcgcttcgacaactgcggcctgacgacttgtcttcgagtcaagggagtgtacactgacgttacacggcacacgcacacacaacaccaccaca

SEQ ID NO: 35 - DNA, 1500 bp, Yarrowia lipolytica
tatttgcgtcgccatacggagaaaccacagtttcagatgacgcggagtagtgggcggtctgagtttggcttccgcacaggatctgtccagttacactttcacgttcttcctttgcatggaattttcttttagctttactcaacaattttggactgtgtagtgggtggacaacaatggagagaatgagacccagaaagtcgtatagcgacacccaagaccgaccagtagctcccatgtaaaatctctgacccaaactcctgtcaatttccttcattactccatgctaaaacgctagcttcggtgttcgttttgcttttttgattttggcttagatttggcccaatgcttagcgaaacgcggggttcccaaacaagacagtaatacactggggagaggacaaaaatcctgacggagcaaagagaagccagctcagaagctattgtgaggttccaaagagaccacatgctgcaggggagaggtgggggagcccgcagagagcacagaagtcacatctggggtctttacaaacaaacagggggtaccctgaatccactgactctggggtatgtccggggtatgcagcccacaggtcagtttagaacgccgtttcaaacgcctgcaaaacgacttttagagccacgagaagactgacttgatacgcaactggagaaacaagaaacaaatacatgtatgtactgctcaaatatcgacattgcacagatgtttcacccttcatacaacacaggtatacacgttcgcagacgctaataaccagctctgcgatcaactctaaccttgtgagtaacccagcaaatgacgattgcggagaagctccagcgggtgtcacgaacggtggaggtggaaaataatggtggtttaaagacataaaattggtagcaacagtgatgaggacacactctaggacgtctggtaccacaaggaggggccaactgtcgctgtcatcgctgtctcctggacagcagagctaactgttgtactccagtgaccaaccaaaattcttctaatgttgcggctcaaggtctgtccccacaactgttgaaagcctaagcgtcatggtaacaacgaggaacaagggcttttcgaaccttgtgcgatgacaacagcatgtgaataagtgttagtggggaagattcaagacagcagaaagttagcgggtgtaagggggggaggaccagagggggtgttaactcatcagaacctttcctgccgagatgtcagcaatcaattcgccttccatacatcttatgatgctatagattccagttctgaggtgttcctggtatgttttcatcttctttcattccattcgagatccctcaagagtgcatgtaaactgaaaccttatgccaaactgagcgatcgtgaatatgaaaaagtctgggaaagcgtcaattcaaaaaagcgaacaaaaaaagcacagaggtatatatataggtgacagcaccaaaccataggtcctccccagaatactcctgcact

SEQ ID NO: 36 - DNA, 1500 bp, Yarrowia lipolytica
agcaacgtcaattgagggtgtgggggaggggcaaggggtgtgattgttggtatggtgcttaatccgaccctgacaaatacacctgtcaagttcgtttcttcctatcttgtctccttgtacttgcaccatctcttgctgtgtctcagacccttacaaccgaagctttgaaagtcgcgcattgttggacagcgaaggcctctgaaacatggactgtcgtagcactcatccaagttgctacttttattctccgatggcgtgttgatactgcacggtatgcacattctcataccgtagcctccgcacaagattgctattatctggttttagcggcaaggtaaaaagcgacgagcgtcgaaacgcgctcaattgcaggcgacagaaaggtttgagctctggttcccaaagttttgtccacaaagagttcgaatttggcccagaaaagttctttatcacaaccttgtcggctttggttgggtgatgatgattggagagagagtggagacggccaaaaaaaaaagaagaaaaaaaaagagaacagtgtggtaccttgagtactgtagagaataaacctccgtggaccaacataggatcaatgtcaaccactgcgttccgtctatagttcaagcaactactcttgtatcggctatagcacggaaatatgtacactacactatgtactccacgttgctaaaggttgcgtgatagttaaatgagaaattgtattttactcacaagcctgttgatggggtagctgccttccggtaatttgttgcggaatcaattgcaatctggactagcgatgtgatgcaacctctggccagggcgttacgcggtccaccttctctgttcaagagcaatacgacttgtgaggtctgcggtctgtttgtttcgtggactcggctacagtaagttttagacacagtttttttgtagttgctgacggtgcagatgagatgcggtccagggtgagattttccggaggatacggtgggccgatgtattccgtgatcccataatgataacacaattctgaaagtcggaatatgtgcgcgtcgccacggggaaaatttttgttcacttttcatccttgtagccccattgttcgtccagaataaggcagacccgttggcaagccccatcgtcaacaaggttatcagacgtcctcatagaatgcgtaatatgcgcaacagttgggctgagtggttgacgtgggtctcgttgttgagaaggtggcgacgatacgtagaggagtgcgattttgaaggtgaaaaagggaataagaccatccacgtgacctcgtaagtcagatccaaaaatctcgttggtttcgcccattttaccccgttattttgtccccctcacttgggtcctactctcgtcgacaacgtcgctttagacttttggcaacaagcttgattgctcccgtctgtcgtttgtgtcggggctcgtcgcctcttataagcttgctcaactccactccactcaactctccatatccca

SEQ ID NO: 37 - protein, 512 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 38 - protein, 513 aa, Marinobacter aquaeolei
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADALILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 39 - protein, 514 aa, Bermanella marisrubri
MSQYSAFSVSQSLKGKHIFLTGVTGFLGKAILEKLLYSVPQLAQIHILVRGGKVSAKKRFQHDILGSSIFERLKEQHGEHFEEWVQSKINLVEGELTQPMFDLPSAEFAGLANQLDLIINSAASVNFRENLEKALNINTLCLNNIIALAQYNVAAQTPVMQISTCYVNGFNKGQINEEVVGPASGLIPQLSQDCYDIDSVFKRVHSQIEQVKKRKTDIEQQEQALIKLGIKTSQHFGWNDTYTFTKWLGEQLLIQKLGKQSLTILRPSIIESAVREPAPGWVEGVKVADALIYAYAKGRVSIFPGRDEGILDVIPVDLVANAAALSAAQLMESNQQTGYRIYQCCSGSRNPIKLKEFIRHIQNVAQARYQEWPKLFADKPQEAFKTVSPKRFKLYMSGFTAITWAKTIIGRVFGSNAASQHMLKAKTTASLANIFGFYTAPNYRFSSQKLEQLVKQFDTTEQRLYDIRADHFDWKYYLQEVHMDGLHKYALADRQELKPKHVKKRKRETIRQAA

SEQ ID NO: 40 - DNA, 1536 bp, Artificial sequence
  Synthetic DNA codon optimized for M. algicola DG893 FAR DNA
atggccacccagcagcagcagaacggtgcatccgcttcgggcgttctggagcagcttagaggcaagcatgtcttgattaccggtactacaggatttctgggaaaggtggttctggagaagctgatccgaaccgtgcctgacatcggtggtattcatctgctgattagaggcaacaagagacatcctgctgccagagaaagattcttgaacgaaatcgcctcttcctctgtgttcgagcggcttagacatgacgacaacgaagcctttgagactttcctggaggagcgtgtgcactgcatcaccggagaagtgaccgagtcgagatttggccttactcctgagcggttccgagcccttgctggccaagtggatgccttcatcaattccgccgcctctgttaacttcagagaggagctggacaaggcactcaagatcaacaccctgtgtctggagaacgtggctgctctggccgaacttaactccgctatggcagtgatccaagtttccacctgttacgtgaacggcaagaactctggacagatcaccgagtccgttatcaagcccgctggcgaatccatccccagatccacagatggctactacgagatcgaggagctggtccaccttctgcaagacaagatctccgacgtgaaggctcgatactctggcaaggtgttggagaagaagctggtggacctgggcatccgagaggcgaacaactacggctggtctgacacctacaccttcaccaaatggctcggagagcagcttctgatgaaagctctgtccggaagatccctgactatcgtgcggccttccatcatcgagtcggctcttgaagagccttctccaggttggatcgagggcgtgaaggttgctgacgccatcatccttgcgtacgccagagagaaggtttcgttgttccccggcaagcgatctggcatcatcgacgttatccccgtggatctggtggccaactctatcattctctctcttgctgaagccctttctggatctggccagcgtagaatctaccaatgttgttctggcggttctaacccgatttctctgggcaagttcatcgactaccttatggccgaagccaagaccaactatgctgcctacgaccagctcttctaccgacgacccaccaagcccttcgtcgctgtgaaccgaaagctgttcgatgttgtcgtgggaggaatgcgagtgcctctttccattgctggcaaggccatgagattggcgggtcagaatcgagaattgaaggttctcaagaaccttgacactactcgatcgctcgctactatctttggattctacactgctcctgactacatcttccggaatgactctctgatggctcttgcttcccgaatgggagaactcgatcgtgtgctgttccctgttgacgctcgacagatcgactggcagctctacttgtgtaagatccacctgggcggcctgaaccgatatgctctgaaagaacgaaagctgtacagccttagagccgctgatacccgaaagaaggctgct

SEQ ID NO: 41 - DNA, 366 bp, Yarrowia lipolytica
agagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaa

SEQ ID NO: 42 - DNA, 250 bp, Artificial sequence
  Synthetic DNA
taaaccagctgcacacgtatccgtggtttatgaccatctgattacgtcaccagttatggggaacgttattgcctctctttctgaacactaaacccagccaatcacagctccaacatatgtaagctaggtgtcccaacagcagccccggggcggtcccgaagttatttaacctccacagatttcgatttataagagcaaaagttccctccaacgctacaaagtcaacacaacaacactacacacacaacat

SEQ ID NO: 43 - DNA, 250 bp, Artificial sequence
  Synthetic DNA
ttcagcatttacgcacatgcccgtggtttatgatcatctcattacgtcaccagttatggggaaccttattgcctctttttctgaactctaaacccagccaattacagctcgaacatatgtaagattggtgtcccaaaagcagccccggtgcggacccgaacttatttagcctccacacattttgatttataagagcaaaggttcccgccaacgctactaggtcaacacaacaacactacacacacaacat

SEQ ID NO: 44 - DNA, 41 bp, Artificial sequence
  Synthetic DNA primer
ctgctgctgggtggccatgattgattagtttgggtgttggt

SEQ ID NO: 45 - DNA, 43 bp, Artificial sequence
  Synthetic DNA primer
gaccatgattacgccaagcttggcaattggtgggatccttttc

SEQ ID NO: 46 - DNA, 43 bp, Artificial sequence
  Synthetic DNA primer
cagcgtttaggggttgcaaagccaattggtgggatccttttca

SEQ ID NO: 47 - DNA, 41 bp, Artificial sequence
  Synthetic DNA primer
ctgctgctgggtggccattgtaactgtggtgtgaatttctc

SEQ ID NO: 48 - DNA, 44 bp, Artificial sequence
  Synthetic DNA primer
gaccatgattacgccaagcttgggtcggtatctcgctcaatgac

SEQ ID NO: 49 - DNA, 43 bp, Artificial sequence
  Synthetic DNA primer
cagcgtttaggggttgcaaagcgtcggtatctcgctcaatgac

SEQ ID NO: 50 - DNA, 39 bp, Artificial sequence
  Synthetic DNA primer
ctgctgctgggtggccattttcgttttgtttggttgtgg

SEQ ID NO: 51 - DNA, 42 bp, Artificial sequence
  Synthetic DNA primer
gaccatgattacgccaagcttgggtttgatgatggcccatga

SEQ ID NO: 52 - DNA, 43 bp, Artificial sequence
  Synthetic DNA primer
cagcgtttaggggttgcaaagccatgacgagcgaattctcaag

SEQ ID NO: 53 - DNA, 38 bp, Artificial sequence
  Synthetic DNA primer
ctgctgctgggtggccattgtcaggtactgtgtggggt

SEQ ID NO: 54 - DNA, 44 bp, Yarrowia lipolytica
gaccatgattacgccaagcttggaaggaaaaccacgtcattgtg

SEQ ID NO: 55 - DNA, 42 bp, Artificial sequence
  Synthetic DNA primer
cagcgtttaggggttgcaaagcgaaaaccacgtcattgtgtg

SEQ ID NO: 56 - DNA, 40 bp, Artificial sequence
  Synthetic DNA primer
ctgctgctgggtggccattgttgtgttggtatgggttgtg

SEQ ID NO: 57 - DNA, 43 bp, Artificial sequence
  Synthetic DNA primer
gaccatgattacgccaagcttgggacaggtgggtcgtcttttg

SEQ ID NO: 58 - DNA, 42 bp, Artificial sequence
  Synthetic DNA primer
cagcgtttaggggttgcaaagcgacaggtgggtcgtcttttg

SEQ ID NO: 59 - DNA, 46 bp, Artificial sequence
  Synthetic DNA primer
ctgctgctgggtggccatgttgtttgtagatgttactgttcaattg

SEQ ID NO: 60 - DNA, 46 bp, Artificial sequence
  Synthetic DNA primer
gaccatgattacgccaagcttggtggactccataacttgacaagag

SEQ ID NO: 61 - DNA, 49 bp, Artificial sequence
  Synthetic DNA primer
cagcgtttaggggttgcaaagccataacttgacaagagggacattaatc

SEQ ID NO: 62 - DNA, 50 bp, Artificial sequence
  Synthetic DNA primer
ctgctgctgggtggccattgttgtttttgtgtaatgaataagagatattc

SEQ ID NO: 63 - DNA, 46 bp, Artificial sequence
  Synthetic DNA primer
gaccatgattacgccaagcttgggttcgtaccagcaccaatgttag

SEQ ID NO: 64 - DNA, 45 bp, Artificial sequence
  Synthetic DNA primer
cagcgtttaggggttgcaaagcgttcgtaccagcaccaatgttag

SEQ ID NO: 65 - DNA, 43 bp, Artificial sequence
  Synthetic DNA primer
ctgctgctgggtggccatatgttgtgtgtgtagtgttgttgtg

SEQ ID NO: 66 - DNA, 41 bp, Artificial sequence
  Synthetic DNA primer
gaccatgattacgccaagcttgggacgtggtaccgaggctg

SEQ ID NO: 67 - DNA, 40 bp, Artificial sequence
  Synthetic DNA primer
cagcgtttaggggttgcaaagcgacgtggtaccgaggctg

SEQ ID NO: 68 - DNA, 40 bp, Artificial sequence
  Synthetic DNA primer
ctgctgctgggtggccattgtgtttgtgtgttggtgtgtc

SEQ ID NO: 69 - DNA, 55 bp, Artificial sequence
  Synthetic DNA primer
gaccatgattacgccaagcttggacaaaaggtagcagaagtatactgtatactca

SEQ ID NO: 70 - DNA, 54 bp, Artificial sequence
  Synthetic DNA primer
cagcgtttaggggttgcaaagcgtagcagaagtatactgtatactcactctttc

SEQ ID NO: 71 - DNA, 39 bp, Artificial sequence
  Synthetic DNA primer
ctgctgctgggtggccatgttcaattggtgtgtttgggt

SEQ ID NO: 72 - DNA, 49 bp, Artificial sequence
  Synthetic DNA primer
gaccatgattacgccaagcttggtgacctcatagaaacaaagttgactg

SEQ ID NO: 73 - DNA, 49 bp, Artificial sequence
  Synthetic DNA primer
cagcgtttaggggttgcaaagcgacctcatagaaacaaagttgactgac

SEQ ID NO: 74 - DNA, 41 bp, Artificial sequence
  Synthetic DNA primer
ctgctgctgggtggccattgtgtgttgagatgttgtgtgtg

SEQ ID NO: 75 - DNA, 49 bp, Artificial sequence
  Synthetic DNA primer
gaccatgattacgccaagcttgggtgtcattttctaagacatttagcga

SEQ ID NO: 76 - DNA, 49 bp, Artificial sequence
  Synthetic DNA primer
cagcgtttaggggttgcaaagcgtgtcattttctaagacatttagcgag

SEQ ID NO: 77 - DNA, 40 bp, Artificial sequence
  Synthetic DNA primer
ctgctgctgggtggccatggtgataaatgtgtggttagac

SEQ ID NO: 78 - DNA, 45 bp, Artificial sequence
  Synthetic DNA primer
gaccatgattacgccaagcttggcattagctagctagagtccagc

SEQ ID NO: 79 - DNA, 47 bp, Artificial sequence
  Synthetic DNA primer
cagcgtttaggggttgcaaagccattagctagctagagtccagcttc

SEQ ID NO: 80 - DNA, 40 bp, Artificial sequence
  Synthetic DNA primer
ctgctgctgggtggccatttttgtgtgtcttggttggatg

SEQ ID NO: 81 - DNA, 41 bp, Artificial sequence
  Synthetic DNA primer
gaccatgattacgccaagcttggagatggtgctgccaggag

SEQ ID NO: 82 - DNA, 39 bp, Artificial sequence
  Synthetic DNA primer
cagcgtttaggggttgcaaagcgatggtgctgccaggag

SEQ ID NO: 83 - DNA, 46 bp, Artificial sequence
  Synthetic DNA primer
ctgctgctgggtggccattttgaatgtagttgtgttgtatgtacga

SEQ ID NO: 84 - DNA, 40 bp, Artificial sequence
  Synthetic DNA primer
gaccatgattacgccaagcttggtttcggcgtgcaaaatc

SEQ ID NO: 85 - DNA, 40 bp, Artificial sequence
  Synthetic DNA primer
cagcgtttaggggttgcaaagcgtgcaaaatcgcacgaac

SEQ ID NO: 86 - DNA, 35 bp, Artificial sequence
  Synthetic DNA primer
ctgctgctgggtggccattgtcggtgttttgaagc

SEQ ID NO: 87 - DNA, 42 bp, Artificial sequence
  Synthetic DNA primer
gaccatgattacgccaagcttggcatttatcgacccatcgac

SEQ ID NO: 88 - DNA, 40 bp, Artificial sequence
  Synthetic DNA primer
cagcgtttaggggttgcaaagcgaccctccccgacatgtc

SEQ ID NO: 89 - DNA, 45 bp, Artificial sequence
  Synthetic DNA primer
ctgctgctgggtggccataacgatttaactgggtaaaataatatg

SEQ ID NO: 90 - DNA, 40 bp, Artificial sequence
  Synthetic DNA primer
gaccatgattacgccaagcttggcaccgacacacggaaag

SEQ ID NO: 91 - DNA, 48 bp, Artificial sequence
  Synthetic DNA primer
cagcgtttaggggttgcaaagccatagatgttactcatgccatggtac

SEQ ID NO: 92 - DNA, 36 bp, Artificial sequence
  Synthetic DNA primer
ctgctgctgggtggccattgtggtggtggtggtggt

SEQ ID NO: 93 - DNA, 46 bp, Artificial sequence
  Synthetic DNA primer
gaccatgattacgccaagcttggactccttccagaaaaatgtgatg

SEQ ID NO: 94 - DNA, 44 bp, Artificial sequence
  Synthetic DNA primer
cagcgtttaggggttgcaaagcctccttccagaaaaatgtgatg

SEQ ID NO: 95 - DNA, 40 bp, Artificial sequence
  Synthetic DNA primer
ctgctgctgggtggccatcgttttgagagtctggtggagt

SEQ ID NO: 96 - DNA, 43 bp, Artificial sequence
  Synthetic DNA primer
gaccatgattacgccaagcttggctcgtcgtcgaccatctctc

SEQ ID NO: 97 - DNA, 42 bp, Artificial sequence
  Synthetic DNA primer
cagcgtttaggggttgcaaagcctcgtcgtcgaccatctctc

SEQ ID NO: 98 - DNA, 41 bp, Artificial sequence
  Synthetic DNA primer
ctgctgctgggtggccatctgtgttagttcggtttgatgtg

SEQ ID NO: 99 - DNA, 45 bp, Artificial sequence
  Synthetic DNA primer
gaccatgattacgccaagcttgggttagtgtacgtaccgagggtg

SEQ ID NO: 100 - DNA, 40 bp, Artificial sequence
  Synthetic DNA primer
ctgctgctgggtggccattgtgttgtgtgttcgaaatgtg

SEQ ID NO: 101 - DNA, 46 bp, Artificial sequence
  Synthetic DNA primer
gaccatgattacgccaagcttgggtatgcagagtgcacccaattag

SEQ ID NO: 102 - DNA, 38 bp, Artificial sequence
  Synthetic DNA primer
ctgctgctgggtggccattgaatgcctgagagtggggt

SEQ ID NO: 103 - DNA, 48 bp, Artificial sequence
  Synthetic DNA primer
gaccatgattacgccaagcttggcaggtctgtgattggttgaaaactg

SEQ ID NO: 104 - DNA, 37 bp, Artificial sequence
  Synthetic DNA primer
ctgctgctgggtggccattgtggtggtgttgtgtgtg

SEQ ID NO: 105 - DNA, 43 bp, Artificial sequence
  Synthetic DNA primer
gaccatgattacgccaagcttgggatggttgctctcaaagctc

SEQ ID NO: 106 - DNA, 39 bp, Artificial sequence
  Synthetic DNA primer
ctgctgctgggtggccatagtgcaggagtattctgggga

SEQ ID NO: 107 - DNA, 46 bp, Artificial sequence
  Synthetic DNA primer
gaccatgattacgccaagcttggcatacggagaaaccacagtttca

SEQ ID NO: 108 - DNA, 39 bp, Artificial sequence
  Synthetic DNA primer
ctgctgctgggtggccattgggatatggagagttgagtg

SEQ ID NO: 109 - DNA, 42 bp, Artificial sequence
  Synthetic DNA primer
gaccatgattacgccaagcttggcaacgtcaattgagggtgt

SEQ ID NO: 110 - protein, 18 aa, Marinobacter algicola
ERLRHDDNEAFETFLEER

SEQ ID NO: 111 - DNA, 23 bp, Artificial sequence
  Synthetic DNA primer
ccaagcttggcgtaatcatggtc

SEQ ID NO: 112 - DNA, 21 bp, Artificial sequence
  Synthetic DNA primer
aacgccaacaggatccgattc

SEQ ID NO: 113 - DNA, 29 bp, Artificial sequence
  Synthetic DNA primer
ttctcctccagtatcatttttctatccgt

SEQ ID NO: 114 - DNA, 27 bp, Artificial sequence
  Synthetic DNA primer
caatatcgacgcagatacacactctca

SEQ ID NO: 115 - DNA, 26 bp, Artificial sequence
  Synthetic DNA primer
acccgataatatcgtccatatggctc

SEQ ID NO: 116 - DNA, 24 bp, Artificial sequence
  Synthetic DNA primer
taaaccagttgcacacgtttccgt

SEQ ID NO: 117 - DNA, 22 bp, Artificial sequence
  Synthetic DNA primer
gagggcggcgctataacgtagt

SEQ ID NO: 118 - DNA, 21 bp, Artificial sequence
  Synthetic DNA primer
ttgagcacggactccaatatg

SEQ ID NO: 119 - DNA, 22 bp, Artificial sequence
  Synthetic DNA primer
gaagcgttgtttttggggcaag

SEQ ID NO: 120 - DNA, 24 bp, Artificial sequence
  Synthetic DNA primer
ggacaatgaatcgatggagacatg

SEQ ID NO: 121 - DNA, 19 bp, Artificial sequence
  Synthetic DNA primer
cagcgaatggcgtcctcca

SEQ ID NO: 122 - DNA, 30 bp, Artificial sequence
  Synthetic DNA primer
agcaatcaaaatacttgcaaataccggtac

SEQ ID NO: 123 - DNA, 28 bp, Artificial sequence
  Synthetic DNA primer
agtgttttttctatccaaaagggggcca

SEQ ID NO: 124 - DNA, 31 bp, Artificial sequence
  Synthetic DNA primer
tttcgtgatctcattcaatgatttctgtatg

SEQ ID NO: 125 - DNA, 27 bp, Artificial sequence
  Synthetic DNA primer
agtgacctctgtgggtctcttttttgt

SEQ ID NO: 126 - DNA, 28 bp, Artificial sequence
  Synthetic DNA primer
gaggggaattctacctttggattgtttc

(19) United States
(12) Patent Application Publication
(10) Pub. No.: US 2012/0164686 A1
(43) Pub. Date: Jun. 28, 2012

(54) YEAST PROMOTERS

(75) Inventors: Douglas A. Hattendorf, Mountain View, CA (US); Antoinette Sero, Foster City, CA (US)

(73) Assignee: Codexis, Inc., Redwood City, CA (US)

(21) Appl. No.: 13/330,324

(22) Filed: Dec. 19, 2011

Related U.S. Application Data
(60) Provisional application No. 61/502,691, filed on Jun. 29, 2011.
(60) Provisional application No. 61/502,697, filed on Jun. 29, 2011.
(60) Provisional application No. 61/427,032, filed on Dec. 23, 2010.

(51) Int. Cl. C12N 15/81; C12P 21/00; C07H 21/04; C12N 1/19

(52) U.S. Cl. 435/69.1; 435/320.1; 435/254.2; 536/24.1

(57) ABSTRACT

The invention relates to recombinant promoters and expression constructs comprising the promoters that may be used to express a protein of interest in yeast, such as Yarrowia lipolytica.

### CROSS-REFERENCE TO RELATED APPLICATIONS

This application claims benefit of priority to U.S. provisional application No. 61/502,691, filed Jun. 29, 2011; U.S. provisional application No. 61/502,697 filed Jun. 29, 2011; and U.S. provisional application No. 61/427,032, filed Dec. 23, 2010; each of which is herein incorporated by reference for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 90834-825281_ST25.TXT, created on Dec. 19, 2011, 93,497 bytes, machine format IBM-PC, MS-Windows operating system, is hereby incorporated by reference in its entirety and for all purposes.

### BACKGROUND OF THE INVENTION

In many commercial applications using recombinant host cells, strong promoters are required to express commercially useful amounts of a desired protein in the cell. Although numerous promoters are known in the art, only a limited number of promoters have been characterized that provide for improved expression of yeast enzymes that are typically expressed at low levels. Accordingly, there is a need for new promoters that control gene expression. The present invention fulfills this and other needs.

### BRIEF SUMMARY OF THE INVENTION

The invention relates, in part, to the identification of promoters for expression of heterologous proteins in yeast. Thus, in one aspect, the invention provides an expression construct comprising a promoter operably linked to a heterologous DNA sequence encoding a protein, wherein the promoter comprises a nucleotide sequence that: (a) has at least 80% identity, at least 85% identity, at least 90% identity, or at least 95% identity to a nucleotide sequence selected from SEQ ID NOS:1 to 36; or at least 75 contiguous nucleotides, or at least 100 contiguous nucleotides or at least 200 contiguous nucleotides of a sequence selected from SEQ ID NOS:1 to 36; or (b) hybridizes under highly stringent conditions to a nucleotide sequence selected from SEQ ID NOS:1 to 36 or a complement thereof. In some embodiments, the promoter comprises at least 80% identity, at least 90% identity, or at least 95% identity to nucleotides 1 to 100 of SEQ ID NO:5; or to nucleotides 1 to 150 of SEQ ID NO:5; or to nucleotides 1 to 200 of SEQ ID NO:5. In some embodiments, the promoter hybridizes under high stringency hybridization conditions to a nucleic acid having a sequence of SEQ ID NO:5 or a complement thereof. In some embodiments, the promoter comprises SEQ ID NO:5. In some embodiments, the promoter comprises at least 80% identity, at least 90% identity, or at least 95% identity to nucleotides 1 to 100 of SEQ ID NO:10; or to nucleotides 1 to 150 of SEQ ID NO:10; or to nucleotides 1 to 200 of SEQ ID NO:10. In some embodiments, the promoter hybridizes under high stringency hybridization conditions to a nucleic acid having a sequence of SEQ ID NO:10 or a complement thereof. In some embodiments, the promoter comprises SEQ ID NO:10. In some embodiments, the promoter comprises at least 80% identity, at least 90% identity, or at least 95% identity to nucleotides 1 to 100 of SEQ ID NO:15; or to nucleotides 1 to 150 of SEQ ID NO:15; or to nucleotides 1 to 200 of SEQ ID NO:15. In some embodiments, the promoter hybridizes under high stringency hybridization conditions to a nucleic acid having a sequence of SEQ ID NO:5 or a complement thereof. In some embodiments, the promoter comprises SEQ ID NO:5.

In some embodiments, the promoter is operably linked to a heterologous DNA sequence encoding an enzyme. In some embodiments, the enzyme is a reductase, a synthase, a dehydrogenase, an esterase, or a cellulase. In some embodiments, the enzyme is a fatty acyl reductase (FAR). In some embodiments, the FAR enzyme is from a Marinobacter species or Oceanobacter species. In some embodiments, the enzyme is from Marinobacter aquaeolei, Marinobacter algicola or Bermanella marisrubri, or is a variant thereof. In some embodiments, the FAR enzyme is a recombinant enzyme.

In additional aspects, the invention further provides an expression cassette comprising an expression construct of the invention, e.g., as described in the preceding paragraph, and a host cell comprising such an expression cassette. In some embodiments, the expression cassette is integrated into a host cell chromosome. In some embodiments, the host cell is a yeast, e.g., an oleaginous yeast such as Yarrowia. In some embodiments, the yeast is Yarrowia lipolytica. In a further aspect, the invention provides a method for producing a protein in such a host cell comprising culturing the host cell under conditions in which the protein is produced in the cell.

### DETAILED DESCRIPTION OF THE INVENTION

Promoters from Yarrowia lipolytica have been identified and characterized. The promoters can be used for the expression of heterologous genes and recombinant protein production in host cells and particularly in yeast, e.g., Yarrowia, host cells. DNA constructs, vectors, cells and methods for protein production are disclosed.

### DEFINITIONS

Unless defined otherwise, technical and scientific terms used herein generally have the same meaning as commonly understood by one of ordinary skill in the art to which this invention belongs.

As used herein, the term “promoter” refers to a DNA sequence, that initiates and facilitates the transcription of an operatively linked gene sequence in the presence of RNA polymerase and transcription regulators. Promoters may include DNA sequence elements that ensure proper binding and activation of RNA polymerase, influence where transcription will start, affect the level of transcription and, in the case of inducible promoters, regulate transcription in response to environmental conditions. Promoters are located 5′ to the transcribed gene. As used herein, a “promoter sequence” may include all or part of the sequence immediately 5′ from the translation start codon. That is, as used herein, the promoter sequence can include the 5′ untranslated region of the mRNA (which may be, in some embodiments, 100-200 bp in length). Most often the core promoter sequences lie within 1-2 kbp of the translation start site, more often within 1 kbp and often within 750 bp, 500 bp or 200 bp, of the translation start site. By convention, the promoter sequence is usually provided as the sequence on the coding strand of the gene it controls. In the present application, “promoter” refers to the various promoters encompassed by the invention, including but not limited to a promoter comprising a nucleic acid sequence of any one of SEQ ID NOS:1 to 36, and functional subsequences and variants of SEQ ID NOS:1-36. Such promoter sequences can be used to express any number of different polypeptides in various yeast host cells, e.g., Yarrowia lipolytica cells, as described herein.

“Promoter activity” refers to the ability of a promoter to drive expression of a protein encoded by a nucleic acid operably linked to the promoter. Promoter activity of a sequence can be assessed by operably linking the sequence to a reporter gene, and determining expression of the reporter. In some embodiments, the reporter can be a fatty acyl reductase (FAR) protein or RNA transcript that is produced from an expression construct comprising the variant promoter operably linked to a polynucleotide sequence encoding FAR, e.g., a FAR polypeptide from M. algicola DG893 (SEQ ID NO:37). FAR expression may be measured using an antibody to the FAR protein, by measuring RNA transcript levels, or using other assays known in the art, including assays disclosed herein (e.g., an assay for fatty alcohol titer production).

In one approach, promoter activity of a variant or functional fragment of a wild-type promoter set forth in SEQ ID NO:1, SEQ ID NO:6, SEQ ID NO:11, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 can be evaluated in Yarrowia lipolytica. In such assays, Y. lipolytica can be cultured in a suitable medium comprising complex sources of nitrogen, salts, and carbon. An exemplary medium is YP medium, which comprises yeast extract, peptone and glucose. In some cases, a variant or functional fragment of a promoter having the sequence of SEQ ID NOS:1-36 is considered to have promoter activity if the promoter is able to drive expression of at least 25%, at least 50%, at least 60%, at least 70%, at least 80%, at least 90%, or at least 95% or greater, of the protein or RNA, e.g., FAR protein, that is produced using a promoter consisting of the sequence of SEQ ID NOS:1-36 when operably linked to a protein encoding a FAR protein, e.g., a FAR polypeptide from M. algicola DG893, for comparison. For example, the level of FAR protein may be measured as described in Example 1. In one embodiment, a variant promoter or functional fragment is considered to have promoter activity if the promoter is able to produce at least 50%, at least 60%, at least 70%, at least 80%, at least 90%, or at least 95% or greater, of the FAR protein produced using the wildtype promoter under the same expression conditions. In some embodiments, a variant promoter or functional fragment has least 50%, or typically at least 60%, at least 70%, at least 80%, at least 90%, or at least 95% or greater, of the promoter activity compared to the promoter from the translation elongation factor-1α (TEF) gene from Yarrowia lipolytica (SEQ ID NO:41; see U.S. Pat. No. 6,265,185).

When two elements, e.g., a promoter and a coding sequence, are said to be “operably linked,” it is meant that the juxtaposition of the two allows them to be in a functionally active relationship. In other words, a promoter is “operably linked” to a coding sequence when the promoter controls the transcription of the coding sequence. A promoter is operably linked to a protein coding sequence when it is located upstream from a coding sequence and when RNA polymerase binding the promoter will transcribe the protein coding sequence. In general, a promoter of SEQ ID NOS:1-36 are contiguous with the protein encoding sequence. In some embodiments, a functional fragment of one of SEQ ID NOS:1-36 is used. In some embodiments, a functional fragment of SEQ ID NOS:1-36 (or a corresponding variant of the functional fragment) is linked to the protein coding in a way that approximately retains the position of the fragment relative to the protein coding sequence. For example, nucleotides 1-100 of SEQ ID NO:10 may be positioned about 150 bases 5′ to the coding sequence of a heterologous protein (e.g. about 100-200 bases upstream).

The term “wild-type promoter sequence” means a promoter sequence that is found in nature, e.g., any one of SEQ ID NOS:1 to 36, or a functional fragment of such a promoter sequence.

The term “variant” with reference to a promoter means a promoter of the invention that comprises one or more modifications such as substitutions, additions or deletions of one or more nucleotides relative to a wild-type sequence. Such variants retain the ability to drive expression of a protein-encoding polynucleotide to which the promoter is operably linked. Variants can be made by genetic manipulation of a wild-type sequence.

The term “wild-type promoter sequence” means a promoter sequence that is found in nature, e.g., any one of SEQ ID NOS:1 to 36, or a functional fragment of such a promoter sequence.

The terms “modifications” and “mutations” when used in the context of substitutions, deletions, insertions and the like with respect to polynucleotides and polypeptides are used interchangeably herein and refer to changes that are introduced by genetic manipulation to create variants from a wild-type sequence.

“Functional fragment” as used herein refers to a promoter that contains a subsequence, usually of at least 25, 50, 75, 100, 150, 200, 250, 300, or 350, or more, contiguous nucleotides relative to a reference sequence such as one of SEQ ID NOs. 1-36 and has promoter activity. Functional fragments typically comprise at least 50%, at least 60%, at least 70%, at least 80%, at least 90%, or at least 95% or greater, of the promoter activity relative to the 1.5 kb promoter sequence of SEQ ID NO:1, SEQ ID NO:6, SEQ ID NO:11, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36.

The term “nucleic acid” “nucleotides” or “polynucleotide” refers to deoxyribonucleotides or ribonucleotides and polymers thereof in either single-stranded or double-stranded form. Except were specified or otherwise clear from context, reference to a nucleic acid sequence encompasses a double stranded molecule.

The term “gene” is used to refer to a segment of DNA that is transcribed. It may include regions preceding and following the protein coding region (5′ and 3′ untranslated sequence) as well as intervening sequences (introns) between individual coding segments (exons).

The term “isolated” as used herein means a compound, protein, cell, nucleic acid sequence or an amino acid sequence that is removed from at least one component with which it is naturally associated. Reference to an “isolated nucleic acid comprising a promoter” or and “isolated promoter” in the context of this invention means that the promoter is not contiguous with the protein-encoding sequence with which the wildtype promoter is naturally associated.

As used herein, the term “recombinant nucleic acid” has its conventional meaning. A recombinant nucleic acid, or equivalently, polynucleotide, is one that is inserted into a heterologous location such that it is not associated with nucleotide sequences that normally flank the nucleic acid as it is found in nature (for example, a nucleic acid inserted into a vector). Likewise, a nucleic acid sequence that does not appear in nature, for example a variant of a naturally occurring gene, is recombinant. A cell containing a recombinant nucleic acid, or protein expressed in vitro or in vivo from a recombinant nucleic acid are also “recombinant.” The term “recombinant” when used with reference to, e.g., a cell, nucleic acid, or polypeptide, thus refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

In the context of this invention, a “reporter protein” refers to any polypeptide gene expression product that is encoded by a heterologous gene operably linked to a promoter of the invention.

The terms “polypeptide,” and “protein” are used interchangeably to refer to a polymer of amino acid residues.

The term “transformed”, in the context of introducing a nucleic acid sequence into a cell, includes introducing a nucleic acid by transfection, transduction or transformation. The nucleic acid sequence may be maintained in the cell as an extrachromosomal element or may be integrated into a chromosome.

The term “expression construct” refers to a polynucleotide comprising a promoter sequence operably linked to a heterologous protein-encoding sequence. The protein is expressed when the expression construct is present in a cell that is cultured under conditions that allow for expression of the protein.

An “expression cassette” as used herein, is a polynucleotide that contains a protein-coding sequence and a promoter and other nucleic acid elements that permit transcription in a host cell (e.g., termination/polyadenylation sequences). An expression cassette is an example of an “expression construct”.

An “expression vector” is a vector comprising an expression construct (such as an expression cassette). An expression vector is also an example of an “expression construct”.

The term “vector,” as used herein, refers to a recombinant nucleic acid designed to carry a coding sequence of interest to be introduced into a host cell. The term “vector” encompasses many different types of vectors, such as cloning vectors, expression vectors, shuttle vectors, plasmids, phage or virus particles, and the like. Vectors include PCR-based vehicles as well as plasmid vectors. Vectors typically include an origin of replication and usually includes a multicloning site and a selectable marker. In some embodiments, a vector comprising a promoter of the invention is used as an integration vector so that the promoter is integrated into a yeast host cell chromosome or into an episomal plasmid present in the yeast strain.

As used herein the term “expression” of a gene means transcription of the gene or, more usually, refers to production of a polypeptide encoded in the gene sequence.

As used herein, the terms “identical” or percent “identity,” in the context of describing two or more polynucleotide sequences, refer to two or more sequences that are the same or have a specified percentage of nucleotides that are the same when compared and aligned for maximum correspondence over a comparison window or designated region, as measured using one of the following sequence comparison algorithms or by manual alignment and visual inspection.

For sequence comparison, typically one sequence acts as a reference sequence, to which test sequences are compared. Alignments and calculation of sequence identity may be done manually (by inspection) but is generally carried out using computer implemented algorithms. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of a wild-type promoter sequence, e.g., any one of SEQ ID NO:1 to SEQ ID NO:36, with its variants, the BLAST and BLAST 2.0 algorithms and the default parameters discussed below may be used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). A “comparison window” as used in alignment algorithms herein, includes reference to a segment of any one of the number of contiguous positions selected from the group consisting of from 20 to 500, usually about 50 to about 300, also about 50 to 250, and also about 100 to about 200 in which a sequence may be compared to a reference sequence of the same number of contiguous positions after the two sequences are optimally aligned.

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “heterologous,” when used to describe a promoter and an operably linked coding sequence, means that the promoter and the coding sequence are not associated with each other in nature. A promoter and a heterologous coding sequence may be from two different organisms. Alternatively, a promoter and a heterologous coding sequence may be from the same organism, provided the particular promoter does not direct the transcription of the coding sequence in the wild-type organism.

A “host cell” in the context of the present invention is a cell into which an expression construct of the present invention may be introduced and expressed. The term encompasses both a cell comprising the expression construct and progeny of such a cell.

A “recombinant host cell” refers to a cell into which has been introduced a heterologous polynucleotide, gene, promoter, e.g., an expression vector, or to a cell having a heterologous polynucleotide or gene integrated into a chromosome or integrated into a naturally occurring episomal plasmid that is present in the host cell.

As used herein “a”, “an”, and “the” include plural references unless the context clearly dictates otherwise.

The term “comprising” and its cognates are used in their inclusive sense; that is, equivalent to the term “including” and its corresponding cognates.

### General Methods

Unless indicated otherwise, the techniques and procedures described or referred to herein are generally performed according to conventional methods well known in the art. Texts disclosing general methods and techniques in the field of recombinant genetics include Sambrook and Russell, Molecular Cloning, A Laboratory Manual (3rd ed. 2001); Ausubel, ed., Current Protocols in Molecular Biology, John Wiley Interscience (1990-2010); each of which incorporated by reference herein, for all purposes. DNA sequences can be obtained by cloning, or by chemical synthesis.

Methods for recombinant expression of proteins in yeast and other organisms are well known in the art, and a number suitable expression vectors are available or can be constructed using routine methods. For example, methods, reagents and tools for transforming yeast are described in “Guide to Yeast Genetics and Molecular Biology,” C. Guthrie and G. Fink, Eds., Methods in Enzymology 350 (Academic Press, San Diego, 2002). Methods, reagents and tools for transforming Y. lipolytica are found in “Yarrowia lipolytica,” C. Madzak, J. M. Nicaud and C. Gaillardin in “Production of Recombinant Proteins. Novel Microbial and Eucaryotic Expression Systems,” G. Gellissen, Ed. 2005. In some embodiments, introduction of the DNA construct or vector of the present invention into a host cell can be effected by calcium phosphate transfection, DEAE-Dextran mediated transfection, electroporation, lithium acetate and polyethylene glycol, or other common techniques.

### Expression Constructs and Uses Thereof

YALI0E12683 promoters

A promoter region of a gene from Yarrowia lipolytica was identified (see Examples below) and is set forth below as SEQ ID NO:1. This promoter region, designated YALI0E12683, is a strong driver of expression in yeast, e.g., Yarrowia lipolytica. A YALI0E12683 promoter sequence can be operably linked to a sequence encoding a heterologous protein, to express the heterologous protein in a host cell.

In some embodiments the YALI0E12683 promoter of the invention will comprise SEQ ID NO:1. In some embodiments the YALI0E12683 promoter comprises a subsequence of SEQ ID NO:1, or a variant thereof, as discussed below. In some embodiments the YALI0E12683 promoter of the invention comprises SEQ ID NO:2, nucleotides 501-1500 of SEQ ID NO:1, which is the 3′ (3-prime) 1 kb of SEQ ID NO:1. In some embodiments the YALI0E12683 promoter of the invention comprises SEQ ID NO:3, nucleotides 751-1500 of SEQ ID NO:1, which is the 3′ (3-prime) 0.75 kb of SEQ ID NO:1. In some embodiments the YALI0E12683 promoter of the invention comprises SEQ ID NO:4, nucleotides 1001-1500 of SEQ ID NO:1, which is the 3′ (3-prime) 0.5 kb of SEQ ID NO:1. In some embodiments the YALI0E12683 promoter of the invention comprises nucleotides SEQ ID NO:5, nucleotides 1251-1500 of SEQ ID NO:1, which is the 3′ (3-prime) 0.25 kb of SEQ ID NO:1.

In some embodiments a YALI0E12683 promoter of the invention comprises a subsequence of SEQ ID NO:1 that retains promoter activity. Subsequences that retain promoter activity are identified using routine methods such as those described hereinbelow. For example, provided with SEQ ID NO:1, or a subsequence thereof, such as SEQ ID NO:2, SEQ ID NO:3, SEQ ID NO:4, or SEQ ID NO:5, any of a number of different functional fragments or variants of the starting sequence can be readily prepared. The promoter activity of a subsequence can be compared to the promoter activity of SEQ ID NO:1. In some embodiments, promoter activity of a subsequent or variant is determined in Yarrowia lipolytica cultured in a nitrogen limitation medium to which exogenous nitrogen is not added.

Constructs containing subsequences of promoter sequences can be made using a variety of routine molecular biological techniques. For illustration and not limitation, SEQ ID NO:1, or a fragment of SEQ ID NO:1, e.g., SEQ ID NO:2, SEQ ID NO:3, SEQ ID NO:4, or SEQ ID NO:5 is cloned into an expression vector so that it is 5′ to, and operably linked to, a sequence encoding a reporter protein. One or a series of deletion constructs may be made to produce one or a library of expression vectors with subsequences of the promoter operably linked to the sequence encoding the reporter protein. Deletions may be made from the 5′ end, the 3′ end or internally. Methods for making deletions include, for illustration and not limitation, using restriction and ligation to remove a portion of the promoter from the vector, using exonucleases to trim the end(s) of the parent sequence, randomly fragmenting the parent sequence and preparing a library of clones containing fragments, or using PCR techniques. The expression vector(s) is then introduced into a host cell and the cell is cultured under conditions in which the protein is produced, with the presence and level of production being indicative of promoter activity. The reporter protein may be one frequently used to assess promoter strength and properties, such as luciferase. Alternatively, the reporter may be another protein, e.g., a yeast protein, such as a Yarrowia lipolytica protein; or an enzyme such as a FAR protein, for example, a FAR protein from M. algicola DG893.

In some embodiments, a YALI0E12683 promoter sequence of the invention will comprise at least 1000 contiguous nucleotides of SEQ ID NO:1, at least 900 nucleotides of SEQ ID NO:1, at least 800 contiguous nucleotides of SEQ ID NO:1, at least 700 contiguous nucleotides of SEQ ID NO:1, at least 600 contiguous nucleotides of SEQ ID NO:1, at least 500 contiguous nucleotides of SEQ ID NO:1, at least 450 contiguous nucleotides of SEQ ID NO:1, at least 400 contiguous nucleotides of SEQ ID NO:1, at least 350 contiguous nucleotides of SEQ ID NO:1, at least 300 contiguous nucleotides of SEQ ID NO:1, at least 250 contiguous nucleotides of SEQ ID NO:1, at least 200 contiguous nucleotides of SEQ ID NO: 1, at least 150 contiguous nucleotides of SEQ ID NO: 1, at least 100 contiguous nucleotides of SEQ ID NO: 1, or at least 75 or at least 50, contiguous nucleotides of SEQ ID NO:1.

In some embodiments, the YALI0E12683 promoter sequence will comprise a subsequence of SEQ ID NO:1 comprising 75 to 1000 contiguous nucleotides of SEQ ID NO:1. In other embodiments the YALI0E12683 promoter sequence will comprise a subsequence of SEQ ID NO:1 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:1. In some embodiments the subsequence comprises at least 25, at least 50, at least 100, at least 150, or at least 200 contiguous nucleotides of the region of SEQ ID NO:5. In some embodiments the subsequence comprises at least 25, at least 50, at least 100, at least 150, at least 200, at least 250, at least 300, at least 350, at least 400, or at least 450 contiguous nucleotides of the region of SEQ ID NO:4. In some embodiments, the fragment may comprise a region of SEQ ID NO:1, SEQ ID NO:2, SEQ ID NO:3, SEQ ID NO:4, or SEQ ID NO:5 that lacks 3′ nucleotides. For example, such a fragment may lack the 3′ 10, 15, 20, 25, 30, 50, 100, or 200 nucleotides from SEQ ID NOs. 1-5 or a variant thereof as described herein.

### YALI0E19206 Promoters

A promoter region of a gene from Yarrowia lipolytica was identified (see Examples below) and is set forth below as SEQ ID NO:6. This promoter region, designated YALI0E19206, is a strong driver of expression in yeast, e.g., Yarrowia lipolytica. A YALI0E19206 promoter sequence can be operably linked to a sequence encoding a heterologous protein, to express the heterologous protein in a host cell.

In some embodiments a YALI0E19206 promoter of the invention will comprise SEQ ID NO:6. In some embodiments the YALI0E19206 promoter comprises a subsequence of SEQ ID NO:6, or a variant thereof, as discussed below. In some embodiments the YALI0E19206 promoter of the invention comprises SEQ ID NO:7, nucleotides 501-1500 of SEQ ID NO:6, which is the 3′ (3-prime) 1 kb of SEQ ID NO:6. In some embodiments the YALI0E19206 promoter of the invention comprises SEQ ID NO:8, nucleotides 751-1500 of SEQ ID NO:6, which is the 3′ (3-prime) 0.75 kb of SEQ ID NO:6. In some embodiments the YALI0E19206 promoter of the invention comprises SEQ ID NO:9, nucleotides 1001-1500, which is the 3′ (3-prime) 0.5 kb of SEQ ID NO:6. In some embodiments the YALI0E19206 promoter of the invention comprises SEQ ID NO:10, nucleotides 1251-1500, which is the 3′ (3-prime) 0.25 kb of SEQ ID NO:6.

In some embodiments a YALI0E19206 promoter of the invention will comprises a subsequence of SEQ ID NO:6 that retains promoter activity. Subsequences that retain promoter activity are identified using routine methods such as those described herein. Provided with SEQ ID NO:6, or a subsequence thereof such as SEQ ID NO:7, SEQ ID NO:8, SEQ ID NO:9, or SEQ ID NO:10, any of a number of different functional deletion mutants of the starting sequence can be readily prepared. The promoter activity of a subsequence can be compared to the promoter activity of SEQ ID NO:6. In some embodiments, promoter activity of a subsequent or variant is determined in Yarrowia lipolytica cultured in a nitrogen limitation medium to which exogenous nitrogen is not added.

Constructs containing subsequences of promoter sequences can be made using a variety of routine molecular biological techniques. For illustration and not limitation, a fragment comprising SEQ ID NO:6, or fragment comprising a subsequence of SEQ ID NO:6, such as SEQ ID NO:7, SEQ ID NO:8, SEQ ID NO:9, or SEQ ID NO:10, may be cloned into an expression vector so that it is 5′ to and operably linked to a heterologous sequence encoding a reporter protein. One or a series of deletion constructs may be made to produce one or a library of expression vectors with subsequences of the promoter operably linked to the sequence encoding the reporter protein. Deletions may be made from the 5′ end, the 3′ end or internally. Methods for making deletions include, for illustration and not limitation, using restriction and ligation to remove a portion of the promoter from the vector, using exonucleases to trim the end(s) of the parent sequence, randomly fragmenting the parent sequence and preparing a library of clones containing fragments, or by using PCR techniques. The expression vector(s) is then introduced into a host cell and the cell is cultured under conditions in which the protein is produced, with the presence and level of production being indicative of promoter activity. The reporter protein may be one frequently used to assess promoter strength and properties, such as luciferase. Alternatively, the reporter may be another protein, e.g., a yeast protein, such as a Yarrowia lipolytica protein; or an enzyme such as a FAR protein, for example, a FAR protein from M. algicola DG893.

In some embodiments, a YALI0E19206 promoter sequence of the invention will comprise at least 1000 contiguous nucleotides of SEQ ID NO:6, at least 900 contiguous nucleotides of SEQ ID NO:6, at least 800 contiguous nucleotides of SEQ ID NO:6, at least 700 contiguous nucleotides of SEQ ID NO:6, at least 600 contiguous nucleotides of SEQ ID NO:6, at least 500 contiguous nucleotides of SEQ ID NO:6, at least 450 contiguous nucleotides of SEQ ID NO:6, at least 400 contiguous nucleotides of SEQ ID NO:6, at least 350 contiguous nucleotides of SEQ ID NO:6, at least 300 contiguous nucleotides of SEQ ID NO:6, at least 250 contiguous nucleotides of SEQ ID NO:6, at least 200 contiguous nucleotides of SEQ ID NO:6, at least 150 contiguous nucleotides of SEQ ID NO:6, at least 100 contiguous nucleotides of SEQ ID NO:6, at least 75, at least 50, contiguous nucleotides of SEQ ID NO:6.

In some embodiments, the YALI0E19206 promoter sequence will comprise a subsequence of SEQ ID NO:6 comprising 75 to 1000 contiguous nucleotides of SEQ ID NO:6. In other embodiments the YALI0E19206 promoter sequence will comprise a subsequence of SEQ ID NO:6 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:6. In some embodiments the subsequence comprises at least 25, at least 50, at least 100, at least 150, or at least 200 contiguous nucleotides of the region of SEQ ID NO:10. In some embodiments the subsequence comprises at least 25, at least 50, at least 100, at least 150, at least 200, at least 250, at least 300, at least 350, at least 400, or at least 450 contiguous nucleotides of SEQ ID NO:9. In some embodiments, the fragment may comprise a region of SEQ ID NO:6, SEQ ID NO:7, SEQ ID NO:8, SEQ ID NO:9 or SEQ ID NO:10 that lacks 3′ nucleotides. For example, such a fragment may lack the 3′ 10, 15, 20, 25, 30, 50, 100, or 200 nucleotides from SEQ ID NOs. 6-10 or a variant as described herein.

### YALI0E34749 Promoters

A promoter region of a gene from Yarrowia lipolytica was identified (see Examples below) and is set forth below as SEQ ID NO:6. This promoter region, designated YALI0E34749 is a strong driver of expression in yeast, e.g., Yarrowia lipolytica. A YALI0E34749 promoter sequence can be operably linked to a sequence encoding a heterologous protein, to express the heterologous protein in a host cell.

In some embodiments a YALI0E34749 promoter of the invention comprises SEQ ID NO:11. In some embodiments the YALI0E34749 promoter comprises a subsequence of SEQ ID NO:11, or a variant thereof, as discussed below. In some embodiments the YALI0E34749 promoter of the invention comprises SEQ ID NO:12, nucleotides 500-1500 of SEQ ID NO:11, which is the 3′ (3-prime) 1 kb of SEQ ID NO:11. In some embodiments the YALI0E34749 promoter of the invention comprises SEQ ID NO:13, nucleotides 751-1500 of SEQ ID NO:11, which is the 3′ (3-prime) 0.75 kb of SEQ ID NO:11. In some embodiments the YALI0E34749 promoter of the invention comprises SEQ ID NO:14, nucleotides 1001-1500 of SEQ ID NO:11, which is the 3′ (3-prime) 0.5 kb of SEQ ID NO:3. In some embodiments the YALI0E34749 promoter of the invention comprises SEQ ID NO:15, nucleotides 1251-1500 of SEQ ID NO:11, which is the 3′ (3-prime) 0.25 kb of SEQ ID NO:11.

In some embodiments a YALI0E34749 promoter of the invention will comprises a subsequence of SEQ ID NO:11 that retains promoter activity. Subsequences that retain promoter activity are identified using routine methods such as those described hereinbelow. Provided with SEQ ID NO:11, or a subsequence thereof, such as SEQ ID NO:12, SEQ ID NO:13, SEQ ID NO:14, or SEQ ID NO:15, any of a number of different functional deletion mutants of the starting sequence can be readily prepared. The promoter activity of a subsequence can be compared to the promoter activity of SEQ ID NO:11. In some embodiments, promoter activity of a subsequent or variant is determined in Yarrowia lipolytica cultured in a nitrogen limitation medium to which exogenous nitrogen is not added.

Constructs containing subsequences of promoter sequences can be made using a variety of routine molecular biological techniques. For illustration and not limitation, a fragment comprising SEQ ID NO:11, or a subsequence of SEQ ID NO:11, such as SEQ ID NO:12, SEQ ID NO:13, SEQ ID NO:14, or SEQ ID NO:15 may be cloned into an expression vector so that it is 5′ to and operably linked to a heterologous sequence encoding a reporter protein. One or a series of deletion constructs may be made to produce one or a library of expression vectors with subsequences of the promoter operably linked to the sequence encoding the reporter protein. Deletions may be made from the 5′ end, the 3′ end or internally. Methods for making deletions include, for illustration and not limitation, using restriction and ligation to remove a portion of the promoter from the vector, using exonucleases to trim the end(s) of the parent sequence, randomly fragmenting the parent sequence and preparing a library of clones containing fragments, using PCR techniques, etc. The expression vector(s) is then introduced into a host cell and the cell is cultured under conditions in which the protein is produced, with the presence and level of production being indicative of promoter activity. The reporter protein may be one frequently used to assess promoter strength and properties, such as luciferase. Alternatively, the reporter may be another protein, e.g., a yeast protein, such as a Yarrowia lipolytica protein; or an enzyme such as a FAR protein, for example, a FAR protein from M. algicola DG893.

In some embodiments, a YALI0E34749 promoter sequence of the invention will comprise at least 1000 contiguous nucleotides of SEQ ID NO:11, at least 900 contiguous nucleotides of SEQ ID NO:11, at least 800 contiguous nucleotides of SEQ ID NO:11, at least 700 contiguous nucleotides of SEQ ID NO:11, at least 600 contiguous nucleotides of SEQ ID NO:11, at least 500 contiguous nucleotides of SEQ ID NO:11, at least 450 contiguous nucleotides of SEQ ID NO:11, at least 400 contiguous nucleotides of SEQ ID NO:11, at least 350 contiguous nucleotides of SEQ ID NO:11, at least 300 contiguous nucleotides of SEQ ID NO:11, at least 250 contiguous nucleotides of SEQ ID NO:11, at least 200 contiguous nucleotides of SEQ ID NO:11, at least 150 contiguous nucleotides of SEQ ID NO:11, at least 100 contiguous nucleotides of SEQ ID NO:11, at least 75 or at least 50, contiguous nucleotides of SEQ ID NO:11.

In some embodiments, the YALI0E34749 promoter sequence comprises a subsequence of SEQ ID NO:11 comprising 75 to 1000 contiguous nucleotides of SEQ ID NO:11. In other embodiments the YALI0E34749 promoter sequence will comprise a subsequence of SEQ ID NO:311 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:11. In some embodiments the subsequence comprises at least 25, at least 50, at least 100, at least 150, or at least 200 contiguous nucleotides of the region of SEQ ID NO:15. In some embodiments the subsequence comprises at least 25, at least 50, at least 100, at least 150, at least 200, at least 250, at least 300, at least 350, at least 400, or at least 450 contiguous nucleotides of SEQ ID NO:14. In some embodiments, the fragment may comprise region of SEQ ID NO:11, SEQ ID NO:12, SEQ ID NO:13, SEQ ID NO:14, or SEQ ID NO:15 that lacks 3′ nucleotides. For example, such a fragment may lack the 3′ 10, 15, 20, 25, 30, 50, or 100 nucleotides from SEQ ID NOs. 11-15 or a variant as described herein.

### Additional Promoters

Additional promoter regions from genes from Yarrowia lipolytica were also identified (see Examples below). These promoter regions, designated YALI0F09185, YALI0B05610, YALI0D14850, YALI0F24673, YALI0E01298, YALI0F07711, YALI0D07634, YALI0B00792, YALI0F16819, YALI0E18568, YALI0F05214, YALI0D16357, YALI0D00627, YALI0D14344, YALI0B02178, YALI0B18150, YALI0C11341, YALI0A21307, YALI0D01441, YALI0E25982, and YALI0B02332, are strong drivers of expression in Yarrowia. Examples of sequences of these promoters are provided in SEQ ID NO:16-36, respectively. Such promoter sequence can be operably linked to a sequence encoding a heterologous protein, to express the heterologous protein in a host cell.

In some embodiments, a promoter of the invention will comprise a sequence selected from SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36. In some embodiments the promoter comprises a subsequence of the selected sequence, or a variant thereof, as discussed below. In some embodiments, the promoter comprises nucleotides 501-1500 of the selected sequence, which is the 3′ 1 kb of the selected sequence. In some embodiments the promoter comprises nucleotides 751-1500 of the selected sequence, which is the 3′ 0.75 kb of the selected sequence. In some embodiments the promoter comprises nucleotides 1001-1500 of the selected sequence, which is the 3′ 0.5 kb of the selected sequence. In some embodiments the promoter comprises nucleotides 1251-1500 of the selected sequence, which is the 3′ 0.25 kb of the selected sequence.

In some embodiments a promoter of the invention comprises a subsequence of a sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 that retains promoter activity. Subsequences that retain promoter activity are identified using routine methods such as those described hereinbelow. For example, provided with SEQ ID NO:16, or a subsequence thereof, any of a number of different functional deletion mutants of the starting sequence can be readily prepared. The promoter activity of a subsequence can be compared to the promoter activity of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36. In some embodiments, promoter activity of a subsequence or variant is determined in Yarrowia lipolytica.

Constructs containing subsequences of promoter sequences can be made using a variety of routine molecular biological techniques. For illustration and not limitation, a sequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36, or a fragment of the selected sequence is cloned into an expression vector so that it is 5′ to, and operably linked to, a sequence encoding a reporter protein. One or a series of deletion constructs may be made to produce one or a library of expression vectors with subsequences of the selected sequence operably linked to the sequence encoding the reporter protein. Deletions may be made from the 5′ end, the 3′ end or internally. Methods for making deletions include, for illustration and not limitation, using restriction and ligation to remove a portion of the sequence from the vector, using exonucleases to trim the end(s) of the parent sequence, randomly fragmenting the parent sequence and preparing a library of clones containing fragments, or using PCR techniques. The expression vector(s) is then introduced into a host cell and the cell is cultured under conditions in which the protein is produced, with the presence and level of production being indicative of promoter activity. The reporter protein may be one frequently used to assess promoter strength and properties, such as luciferase. Alternatively, the reporter may be another protein, e.g., a yeast protein, such as a Yarrowia lipolytica protein; or an enzyme such as a FAR protein, for example, a FAR protein from M. algicola DG893.

In some embodiments, a promoter sequence of the invention will comprise at least 1000 contiguous nucleotides, at least 900 contiguous nucleotides, at least 800 contiguous nucleotides, at least 700 contiguous nucleotides, at least 600 contiguous nucleotides, at least 500 contiguous nucleotides, at least 450 contiguous nucleotides, at least 400 contiguous nucleotides, at least 350 contiguous nucleotides, at least 300 contiguous nucleotides, at least 250 contiguous nucleotides, at least 200 contiguous nucleotides, at least 150 contiguous nucleotides, at least 100 contiguous nucleotides, or at least 75 or at least 50 contiguous nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36.

In some embodiments, a promoter sequence of the invention will comprise a subsequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 comprising 75 to 1000 contiguous nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36. In other embodiments the YALI0E12683 promoter sequence will comprise a subsequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36.

In some embodiments, the fragment may comprise region of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36, or a subsequence thereof, that lacks 3′ nucleotides. For example, such a fragment may lack the 3′ 10, 15, 20, 25, 30, 50, 100, or 200 nucleotides from SEQ ID NOs. 11-15 or a variant as described herein.

### Promoter Variants

As discussed above and elsewhere herein, it is understood that the promoters of this invention may have sequences that are variants of the promoter sequences set forth in SEQ ID NOS. 1-36, or subsequences thereof. In some embodiments, a promoter of the invention can be characterized by its ability to hybridize under high stringency hybridization conditions to a promoter sequence set forth in any one of SEQ ID NOS 1-36, or the complement of the sequence. High stringency hybridization conditions in the context of this invention refers to hybridization at about 5° C. to 10° C. below the melting temperature (T_{M}) of the hybridized duplex sequence, followed by washing at 0.2×SSC/0.1% SDS at 37° C. for 45 minutes. The melting temperature of the nucleic acid hybrid can be calculated as taught by Berger and Kimmel, 1987, Guide to Molecular Cloning Techniques, Methods in Enzymology, Vol. 152, Academic Press, San Diego, Calif.

A promoter of the invention can be characterized based on alignment with one of the sequences described herein, e.g., any one of the sequence set forth in SEQ ID NOs 1 to 36.

In some embodiments, promoters of the invention include sequences with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to SEQ ID NO: 1 or to promoter subsequences described herein having promoter activity, such as SEQ ID NOs 2, 3, 4, or 5. Thus, in some embodiments the promoter has a sequence that has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to a subsequence of SEQ ID NO:1 comprising 75 to 1000 contiguous nucleotides of SEQ ID NO:1, or a subsequence of SEQ ID NO:1 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:1. For example, the promoter sequence may have at least 90%, at least 93%, at least 95%, or at least 98% sequence identity to SEQ ID NO:3, or a subsequence of at least 100, at least 200, at least 300, at least 400, at least 500, at least 600, or at least 700 nucleotides of SEQ ID NO:3. In another example, the promoter sequence may have at least 90%, at least 93%, at least 95%, or at least 98% sequence identity to SEQ ID NO:4, or a subsequence of at least 100, at least 200, at least 300, at least 400 nucleotides of SEQ ID NO:4. In another example, the promoter sequence may have at least 90%, at least 93%, at least 95%, or at least 98% sequence identity to SEQ ID NO:5, or a subsequence of at least 100 or at least 200 nucleotides of SEQ ID NO:5. In some embodiments, the promoter sequence may have at least 80%, at least 85%, at least 90%, or at least 95% sequence identity to a subsequence of SEQ ID NO:5 that lacks the 3′ 50 nucleotides or that lacks the 3′ 100 nucleotides, or the 3′ 150 nucleotides of SEQ ID NO:5.

In some embodiments the promoter comprises a sequence of at least 100 nucleotides that differs from the corresponding subsequence of SEQ ID NO:1 at one or more (e.g., 1, 2, 3, 4, 5, 6, 7, 8, 9, 10, 20, 25, 30, or 40) nucleotides. In some embodiments, the subsequence of SEQ ID NO:1 is SEQ ID NO:2, SEQ ID NO:3, SEQ ID NO:4, or SEQ ID NO:5.

In some embodiments, promoters of the invention include sequences with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to SEQ ID NO: 6 or to promoter subsequences described herein having promoter activity, such as SEQ ID NOs 7, 8, 9, or 10. Thus, in some embodiments the promoter has a sequence that has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to a subsequence of SEQ ID NO:6 comprising 75 to 1000 contiguous nucleotides of SEQ ID NO:6, or a subsequence of SEQ ID NO:6 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:6. For example, the promoter sequence may have at least 90%, at least 93%, at least 95%, or at least 98% sequence identity to SEQ ID NO:8, or a subsequence of at least 100, at least 200, at least 300, at least 400, at least 500, at least 600, or at least 700 nucleotides of SEQ ID NO:8. In another example, the promoter sequence may have at least 90%, at least 93%, at least 95%, or at least 98% sequence identity to SEQ ID NO:9, or a subsequence of at least 100, at least 200, at least 300, at least 400 nucleotides of SEQ ID NO:9. In another example, the promoter sequence may have at least 90%, at least 93%, at least 95%, or at least 98% sequence identity to SEQ ID NO:10, or a subsequence of at least 100 or at least 200 nucleotides of SEQ ID NO:10. In some embodiments, the promoter sequence may have at least 80%, at least 85%, at least 90%, or at least 95% sequence identity to a subsequence of SEQ ID NO:10 that lacks the 3′ 50 nucleotides or that lacks the 3′ 100 nucleotides, or the 3′ 150 nucleotides of SEQ ID NO:10.

In some embodiments the promoter comprises a sequence of at least 100 nucleotides that differs from the corresponding subsequence of SEQ ID NO:6 at one or more (e.g., 1, 2, 3, 4, 5, 6, 7, 8, 9, 10, 20, 25, 30, or 40) nucleotides. In some embodiments, the subsequence of SEQ ID NO:6 is SEQ ID NO:7, SEQ ID NO:8, SEQ ID NO:9, or SEQ ID NO:10.

In some embodiments, promoters of the invention include sequences with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to SEQ ID NO:11 or to subsequences described herein having promoter activity, such as SEQ ID NOs 12, 13, 14, or 15. Thus, in some embodiments the promoter has a sequence that has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to a subsequence of SEQ ID NO:11 comprising 75 to 1000 contiguous nucleotides of SEQ ID NO:11, or a subsequence of SEQ ID NO:11 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides SEQ ID NO:11. For example, the promoter sequence may have at least 90%, at least 93%, at least 95%, or at least 98% sequence identity to SEQ ID NO:13, or a subsequence of at least 100, at least 200, at least 300, at least 400, at least 500, at least 600 or at least 700 nucleotides of SEQ ID NO:13. In another example, the promoter sequence may have at least 90%, at least 93%, at least 95%, or at least 98% sequence identity to SEQ ID NO:14, or a subsequence of at least 100, at least 200, at least 300, at least 400 nucleotides of SEQ ID NO:14. In another example, the promoter sequence may have at least 90%, at least 93%, at least 95%, or at least 98% sequence identity to SEQ ID NO:15, or a subsequence of at least 100 or at least 200 nucleotides of SEQ ID NO:15. In some embodiments, the promoter sequence may have at least 80%, at least 85%, at least 90%, or at least 95% sequence identity to a subsequence of SEQ ID NO:15 that lacks the 3′ 50 nucleotides or that lacks the 3′ 100 nucleotides, or the 3′ 150 nucleotides of SEQ ID NO:15.

In some embodiments the promoter comprises a sequence of at least 100 nucleotides that differs from the corresponding subsequence of SEQ ID NO:11 at one or more (e.g., 1, 2, 3, 4, 5, 6, 7, 8, 9, 10, 20, 25, 30 or 40) nucleotides. In some embodiments, the subsequence of SEQ ID NO:121 is SEQ ID NO:12, SEQ ID NO:13, SEQ ID NO:14, or SEQ ID NO:15.

In some embodiments, promoters of the invention include sequences with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, and at least 99% sequence identity to a sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 or to subsequences that have promoter activity. Thus, in some embodiments the promoter has a sequence that has at least 60%, at least 65%, at least 70%, at least 75%, t at least 80%, at least 85%, at least 90%, at least 91%, at least 92%, at least 93%, at least 94%, at least 95%, at least 96%, at least 97%, at least 98%, and at least 99% sequence identity to a subsequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 comprising 75 to 1000 contiguous nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36; or to a subsequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 comprising 50 to 700, 50 to 600, 50 to 500, 50 to 400, 50 to 300, 50 to 200; 75 to 700, 75 to 600, 75 to 500, 75 to 400, 75 to 300, 75 to 200, 100 to 700, 100 to 600, 100 to 500, 100 to 400, 100 to 300, or 100 to 200 contiguous nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36. For example, the promoter sequence may have at least 90%, at least 93%, at least 95%, or at least 98% sequence identity to SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36, or a subsequence of at least 100, at least 200, at least 300, at least 400, at least 500, or at least 600 nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36. In another example, the promoter sequence may have at least 80%, at least 85%, at least 90%, or at least 95% sequence identity to SEQ ID NO:5, or a subsequence of at least 100 or at least 200 nucleotides of SEQ ID NO:5. In some embodiments, the promoter sequence may have at least 80%, at least 85%, at least 90%, or at least 95% sequence identity to a subsequence, e.g., a subsequence of from 200 to 500 nucleotides in length of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 that lacks the 3′ 50 nucleotides or that lacks the 3′ 100 nucleotides, or the 3′ 150 nucleotides of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36.

In some embodiments the promoter comprises a sequence of at least 100 nucleotides that differs from the corresponding subsequence of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 at one or more (e.g., 1, 2, 3, 4, 5, 6, 7, 8, 9, 10, 20, 25, 30 or 40) nucleotides.

### Production of Variant Promoter Sequences

Provided with the promoter sequences SEQ ID NO:1, SEQ ID NO:6, SEQ ID NO:11, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36 and subsequences disclosed herein, for example, any of a number of different functional variant sequences can be readily prepared and screened for function. For example, mutagenized promoters can be obtained using standard mutagenesis techniques and, optionally, directed evolution methods can be readily applied to polynucleotides such as, for example, the wild-type promoter sequence (e.g., SEQ ID NO:1, SEQ ID NO:6, SEQ ID NO:11, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:25, or SEQ ID NO:36). Mutagenesis may be performed in accordance with any of the techniques known in the art, including random and site-specific mutagenesis. See, for example Ling, et al., “Approaches to DNA mutagenesis: an overview,” Anal. Biochem., 254(2):157-78 (1997); Hemsley et al., “A simple method for site-directed mutagenesis using the polymerase chain reaction.” Nucleic Acids Res. 17(16): 6545-51 (1989); and Matsmura, et al., “Optimization of heterologous gene expression for in vitro evolution.” Biotechniques 30(3): 474-6 (2001). Other general references include the following Dale, et al., “Oligonucleotide-directed random mutagenesis using the phosphorothioate method,” Methods Mol. Biol., 57:369-74 (1996); Smith, “In vitro mutagenesis,” Ann. Rev. Genet., 19:423-462 (1985); Botstein, et al., “Strategies and applications of in vitro mutagenesis,” Science, 229:1193-1201 (1985); Carter, “Site-directed mutagenesis,” Biochem. J., 237:1-7 (1986); Kramer, et al., “Point Mismatch Repair,” Cell, 38:879-887 (1984); Wells, et al., “Cassette mutagenesis: an efficient method for generation of multiple mutations at defined sites,” Gene, 34:315-323 (1985); Minshull, et al., “Protein evolution by molecular breeding,” Current Opinion in Chemical Biology, 3:284-290 (1999); Christians, et al., “Directed evolution of thymidine kinase for AZT phosphorylation using DNA family shuffling,” Nature Biotechnology, 17:259-264 (1999); Crameri, et al., “DNA shuffling of a family of genes from diverse species accelerates directed evolution,” Nature, 391:288-291; Crameri, et al., “Molecular evolution of an arsenate detoxification pathway by DNA shuffling,” Nature Biotechnology, 15:436-438 (1997); Zhang, et al., “Directed evolution of an effective fucosidase from a galactosidase by DNA shuffling and screening,” Proceedings of the National Academy of Sciences, U.S.A., 94:45-4-4509; Crameri, et al., “Improved green fluorescent protein by molecular evolution using DNA shuffling,” Nature Biotechnology, 14:315-319 (1996); Stemmer, “Rapid evolution of a protein in vitro by DNA shuffling,” Nature, 370:389-391 (1994); Stemmer, “DNA shuffling by random fragmentation and reassembly: In vitro recombination for molecular evolution,” Proceedings of the National Academy of Sciences, U.S.A., 91:10747-10751 (1994); WO 95/22625; WO 97/0078; WO 97/35966; WO 98/27230; WO 00/42651; and WO 01/75767. The promoter activity of the variant can be assessed by any suitable method using an appropriate host cell as described herein.

One targeted method for preparing variant promoters relies upon the identification of putative regulatory elements within the target sequence by, for example, comparison with promoter sequences known to be expressed in a similar manner. Sequences which are shared are likely candidates for the binding of transcription factors and are thus likely elements which confer expression patterns. Confirmation of such putative regulatory elements can be achieved by deletion analysis of each putative regulatory region followed by function analysis of each deletion construct by assay of a reporter gene which is functionally attached to each construct.

### Expressed Proteins

To produce a vector such as an expression cassette utilizing the promoters of this invention for gene expression, a variety of methods well known in the art may be used to obtain the polynucleotide sequences for the promoter and a coding sequence of interest, and join the two sequences so that they are operably linked for gene expression. The polypeptide coding sequence may encode a detectable protein including proteins of interest for production and conventional reporter proteins for routine screening for promoter activity.

A promoter of the invention can be used to express any number of proteins in yeast, e.g., Yarrowia. In some embodiments, the coding sequence to which a promoter of the invention is operably linked encodes for a protein such as an enzyme, a therapeutic protein, a receptor protein and the like. In some embodiments, the coding sequence operably linked to a promoter of the invention encodes an enzyme such as cellulases, an aminopeptidases, amylases, carbohydrases, carboxypeptidases, catalases, chitinases, cutinases, cyclodextrin glycosyltransferases, deoxyribonucleases, esterases, α-galactosidases, β-glucanases, β-galactosidases, glucoamylases, α-glucosidases, β-glucosidases, invertases, isomerases, laccases, lipases, mannosidases, mutanases, oxidases, pectinolytic enzymes, peroxidases, phospholipases, phytases, polyphenoloxidases, reductases, transferases, xylanases, or proteolytic enzymes. In some embodiments, the enzyme is a fatty acid synthase, a thioesterase, an acyl-CoA synthase, an an alcohol dehydrogenase, an alcohol acyltransferase, a fatty acid (carboxylic acid) reductase, an acyl-ACP reductase, a fatty acid hydroxylase, an acyl-CoA desaturase, an acyl-ACP desaturase, an acyl-CoA oxidase, an acyl-CoA dehydrogenase, or another enzyme involved in fatty acid metabolism.

A non-limiting representative list of families or classes of enzymes which may be encoded by an expression construct comprising a promoter of the invention includes the following: oxidoreductases (E.C.1); transferases (E.C.2); hydrolyases (E.C.3); lyases (E.C.4); isomerases (E.C. 5) and ligases (E.C. 6). More specific but non-limiting subgroups of oxidoreductases include dehydrogenases (e.g., alcohol dehydrogenases (carbonyl reductases), xylulose reductases, aldehyde reductases, farnesol dehydrogenase, lactate dehydrogenases, arabinose dehydrogenases, glucose dehyrodgenases, fructose dehydrogenases, xylose reductases and succinate dehyrogenases) oxidases (e.g., glucose oxidases, hexose oxidases, galactose oxidases and laccases), monoamine oxidases, lipoxygenases, peroxidases, aldehyde dehydrogenases, reductases, long-chain acyl-[acyl-carrier-protein] reductases, acyl-CoA dehydrogenases, ene-reductases, synthases (e.g., glutamate synthases), nitrate reductases, mono and di-oxygenases, and catalases. More specific but non-limiting subgroups of transferases include methyl, amidino, carboxyl, and phoso-transferases, transketolases, transaldolases, acyltransferases, glycosyltransferases, transaminases, transglutaminases and polymerases. More specific but non-limiting subgroups of hydrolases include invertases, ester hydrolases, peptidases, glycosylases, amylases, cellulases, hemicellulases, xylanases, chitinases, glucosidases, glucanases, glucoamylases, acylases, galactosidases, pullulanases, phytases, lactases, arabinosidases, nucleosidases, nitrilases, phosphatases, lipases, phospholipases, proteases, ATPases, and dehalogenases. More specific but non-limiting subgroups of lyases include decarboxylases, aldolases, hydratases, dehydratases (e.g., carbonic anhydrases), synthases (e.g., isoprene, pinene and farnesene synthases), pectinases (e.g., pectin lyases) and halohydrin dehydrogenases. More specific, but non-limiting subgroups of isomerases include racemases, epimerases, isomerases (e.g., xylose, arabinose, ribose, glucose, galactose and mannose isomerases), tautomerases, and mutases (e.g. acyl transferring mutases, phosphomutases, and aminomutases. More specific but non-limiting subgroups of ligases include ester synthases.

Some non-limiting preferred enzymes include the following cellulases (such as cellobiohydrolases, endoglucanases, beta-glucosidases), invertases, xylanases, hemicellulases, GH61 family proteins, proteases, amylases, xylose, arabinose, and glucose isomerases, reductases (such as xylulose reductases, fatty alcohol reductases, and acyl-CoA reductases); and enzymes that can act as selectable markers, e.g., hygromycin phosphotransferase.

In some embodiments, the coding sequence that is operably linked to the promoter of the invention encodes a protein other than an enzyme, for example the protein may include, hormones, receptors, growth factors, antigens and antibodies (e.g., antibody heavy and light chains). The protein coding sequences operably linked to a promoter of the invention may be chimeric or fusion proteins. Further, the protein coding sequence may include epitope tags (e.g., c-myc, HIS6 or maltose-binding protein) to aid in purification.

In some embodiments, a recombinant expression construct comprising a protein-coding sequence operably linked to a promoter of the invention has an endogenous Yarrowia gene as the protein-encoding sequence.

In some embodiments, a promoter of the invention may be linked to a nucleic acid that encodes a conventional or commercially available reporter protein that is a heterologous protein that has an easily measured activity such as β-galatosidase (lacZ), β-glucuronidase (GUS), fluorescent protein (GFP), luciferase, chloramphenicol, or acetyl transferase (CAT). Any protein for which expression can be measured (e.g., by enzymatic, immunological or physical methods) can serve as a reporter. Although conventional reporters are better suited to high throughput screening, production of any protein can be assayed by immunological methods, mass spectroscopy, etc. Alternatively, expression can be measured at the level of transcription by assaying for production of specific RNAs.

In some embodiments, the sequence of interest to be expressed that is operably linked to a promoter of the invention encodes an enzyme involved in fatty alcohol production. Enzymes that convert fatty acyl-thioester substrates (e.g., fatty acyl-CoA or fatty acyl-ACP) to fatty alcohols are commonly referred to as fatty alcohol forming acyl-CoA reductases or fatty acyl reductases (“FARs”). The terms “fatty alcohol forming acyl-CoA reductase” or “fatty acyl reductase” is used interchangeably herein refers to an enzyme that catalyzes the reduction of a fatty acyl-CoA, a fatty acyl-ACP, or other fatty acyl thioester complex to a fatty alcohol, which is linked to the oxidation of NAD(P)H to NAD(P)+.

Examples of FAR enzymes and nucleic acids encoding such FAR enzymes are provided, e.g., in U.S. Patent Application Publication No. 20110000125, incorporated by reference herein. In some particular embodiments, the enzyme is a FAR enzyme from a Marinobacter species, e.g., M. algicola (strain DG893) (“FAR_Maa”) or M. aquaeolei VT8 (“FAR_Maq”); M. arcticus, M. actinobacterium, and M. lipolyticus; or an Oceanobacter species, e.g., strain RED65 (recently reclassified as Bermanella marisrubri) Oceanobacter strain WH099, and O. kriegii. For example, in some embodiments, the FAR protein is FAR_Maa (SEQ ID NO:37), FAR_Maq (SEQ ID NO:38) or FAR_Ocs (Oceanobacter sp. RED65, SEQ ID NO:39), or a functional variant thereof.

Other examples of FAR enzymes that can be expressed using the promoters of the invention include FAR enzymes from Bombyx mori (see, e.g., Moto et al., 2003, Proc. Nat'l Acad. Sci. USA 100(16):9156-9161) and Arabidopsis thaliana. In other embodiments, the FAR enzyme or variant FAR enzyme is from Vitis vinifera (GenBank Accession No. CA022305.1 or CAO67776.1), Desulfatibacillum alkenivorans (GenBank Accession No. NZ_ABII01000018.1), Stigmatella aurantiaca (NZ_AAMD01000005.1), or Phytophthora ramorum (GenBank Accession No.: AAQX01001105.1). In some embodiments, the FAR enzyme is FAR_Hch (Hahella chejuensis KCTC 2396, GenBank No. YP_{—}436183.1), FAR_JVC (JCVI_ORF_{—}1096697648832, GenBank No. EDD40059.1), FAR_Fer (JCVI_SCAF_{—}1101670217388), FAR_Key (JCVI_SCAF_{—}1097205236585), FAR_Gal (JCVI_SCAF_{—}1101670289386), or a functional variant thereof.

In some embodiments, a promoter of the invention, e.g., having a sequence as set forth in any one of SEQ ID NO:1-36, or a subsequence having promoter activity, may thus be used to drive expression of a FAR protein. Expression of the FAR protein may be measured using an antibody to the FAR protein, or may be assessed using an alternative assay that measures enzyme activity, e.g., an assay such as that described in the examples section that measure fatty alcohol titer. For example, fatty alcohols secreted into the medium can be isolated by solvent extraction of the aqueous medium with a suitable water immiscible solvent. Phase separation followed by solvent removal provides the fatty alcohol which may then be further purified and fractionated using methods and equipment known in the art. For example, extraction can be performed with isopropanol:hexane (4:6 ratio). The extract is centrifuged, the upper organic phase transferred into a vial and analyzed using gas chromatography.

### Expression Constructs

A promoter sequence of the invention and a coding sequence may be operably linked in an expression construct (e.g., an expression vector). A number of known methods are suitable for the purpose of ligating the two sequences, such as ligation methods based on PCR and ligation methods mediated by various ligases (e.g., bacteriophase T4 ligase). The promoter used to direct expression of a heterologous sequence is optionally positioned about the same distance from the heterologous translation start site as it is from the translation start site in its natural setting. As is known in the art, however, some variation in this distance can be accommodated without loss of promoter function. In some embodiments in which there may be a 3′ or internal deletion in a promoter relative to a sequence described herein, such as any one of SEQ ID NOs:1 to 36, maintaining the same distance to the heterologous translation start site can be accomplished by inserting a number of nucleotides approximately equal to the number deleted (e.g., inserting from 70-130% of the number deleted, sometimes 80-120% and sometimes 90-110% of the number of nucleotides deleted). It will be appreciated that a vector comprising a promoter sequence of the invention may comprise flanking sequences (additional nucleotides) 5′ to the promoter sequence and 3′ to the protein coding sequence.

When a promoter sequence of the invention is not truncated at the 3′ end (for example, the promoter is a sequence selected from SEQ ID NOs:1-36, in some embodiments, the promoter sequence may be linked to the protein coding sequence at or close to the translation start codon (e.g., the 5′-UTR of the heterologous gene is deleted). In other embodiments, all or a portion of the 5′-UTR of the heterologous gene to be expressed is retained and a 3′ portion of the promoter may be deleted. In such an embodiment, approximately the same spacing between upstream promoter elements and the translation start site is maintained. This may be considered and example of a promoter operably linked to a protein-encoding sequence.

In addition to the promoter, the expression cassette optionally contains all the additional elements required for the expression of the heterologous sequence in host cells, such as signals required for efficient polyadenylation of the transcript, translation termination, and optionally enhancers. If genomic DNA is used as the heterologous coding sequence, introns with functional splice donor and acceptor sites may also be included. See, e.g., Ausubel et al., Current Protocols in Molecular Biology 1995, including supplements, incorporated herein by reference.

The expression construct can be contained in an expression vector that also includes a replicon that functions in yeast or other host cells, and may contain a gene encoding a selectable marker to permit selection of microorganisms that harbor recombinant vectors. Selectable markers are well known and widely used in the art and include antibiotic resistance genes, metabolic selection markers, and the like. Examples of selectable markers for use in yeast include are resistance to kanamycin, hygromycin and the aminoglycoside G418, as well as ability to grow on media lacking uracil or leucine.

In addition to episomal DNA based expression, the expression construct comprising a promoter of the invention and a polypeptide coding sequence may be integrated into the host DNA, e.g., a host cell chromosome, by homologous recombination. In alternative embodiments, the expression construct may be randomly integrated into the host DNA, e.g., by non-homologous recombination. In some embodiments, a promoter of the invention is introduced into a plasmid harboring a DNA fragment encoding a protein sequence of interest, e.g., a FAR enzyme, for targeted integration into the host cell DNA, e.g., a chromosome, at a desired site. Methods of targeted integration are known (see, e.g., Gaillardin C and Ribet A M (1987) “LEU2 directed expression of β-galactosidase activity and phleomycin resistance in Yarrowia lipolytica.” Current Genetics 11: 369-375).

### Host Cells

In certain embodiments, the recombinant host cell comprising a promoter of the invention operably linked to a heterologous nucleic acid encoding a protein, e.g., a FAR, is a yeast. In various embodiments, the yeast host cell is a species of a genus selected from the group consisting of Candida, Hansenula, Saccharomyces, Schizosaccharomyces, Pichia, Kluyveromyces, Rhodotorula, and Yarrowia. In some embodiments, the yeast host cell is a species of a genus selected from the group consisting of Saccharomyces, Candida, Pichia and Yarrowia.

In various embodiments, the yeast host cell is selected from the group consisting of Hansenula polymorphs, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia ferniemtans, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, Candida krusei, Candida ethanolic and Yarrowia lipolytica and synonyms or taxonomic equivalents thereof. In some embodiments, the host cell is Yarrowia lipolytica. Yarrowia lipolytica is available, as a non-limiting example, from the ATCC under accession numbers 20362, 18944, and 76982.

In certain embodiments, the yeast host cell is a wild-type cell. In various embodiments, the wild-type yeast cell strain is selected from, but not limited to, strain BY4741, strain FL100a, strain INVSC1, strain NRRL Y-390, strain NRRL Y-1438, strain NRRL YB-1952, strain NRRL Y-5997, strain NRRL Y-7567, strain NRRL Y-1532, strain NRRL YB-4149 and strain NRRL Y-567. In other embodiments, the yeast host cell is genetically modified. Examples of genetically modified yeast useful as recombinant host cells include, but are not limited to, genetically modified yeast found in the Open Biosystems collection found at the http www site openbiosystems.com/GeneExpression/Yeast/YKO/. See, Winzeler et al. (1999) Science 285:901-906.

In other embodiments, the recombinant host cell is an oleaginous yeast. Oleaginous yeasts are organisms that accumulate “oil” as a major part of total lipids. The “oil” is composed primarily of triacylglycerols, but may also contain other neutral lipids, phospholipids and free fatty acids. Examples of oleaginous yeast include, but are not limited to, organisms selected from the group consisting of Yarrowia lipolytica, Yarrowia paralipolytica, Candida revkaufi, Candida pulcherrima, Candida tropicalis, Candida utilis, Candida curvata D, Candida curvata R, Candida diddensiae, Candida boldinii, Rhodotorula glutinous, Rhodotorula graminis, Rhodotorula mucilaginosa, Rhodotorula minuta, Rhodotorula bacarum, Rhodosporidium toruloides, Cryptococcus (terricolus) albidus var. albidus, Cryptococcus laurentii, Trichosporon pullans, Trichosporon cutaneum, Trichosporon cutancum, Trichosporon pullulans, Lipomyces starkeyii, Lipomyces lipoferus, Lipomyces tetrasporus, Endomycopsis vernalis, Hansenula ciferri, Hansenula saturnus, and Trigonopsis variabilis. In some embodiments, the oleaginous yeast is Rhodotorula or Yarrowia (e.g. Y. lipolytica). In certain embodiments, Yarrowia lipolytica strains include, but are not limited to DSMZ (Deutsche Sammlung von Mikroorganismen and Zellkulturen GmbH; German Collection of Microorganisms and Cell Cultures) strains DSMZ 1345, DSMZ 3286, DSMZ 8218, DSMZ 70561, DSMZ 70562, DSMZ 21175, and also strains available from the Agricultural Research Service (NRRL) such as but not limited to NRRL YB-421, NRRL YB-423, NRRL YB-423-12 and NRRL YB-423-3. In certain embodiments, the oleaginous yeast is a wild-type organism. In other embodiments, the oleaginous yeast is genetically modified.

Culture of Organisms Transformed with an Expression Construct Comprising a Promoter of the Invention.

Yeast cell culture conditions are well known in the art. Cell culture media in general are set forth in Atlas and Parks, eds., 1993, The Handbook of Microbiological Media. The individual components of media for cultivating yeast cells are available from commercial sources, e.g., under the Difco™ and BBL™ trademarks.

A host cell, e.g., Y. lipolytica, comprising a promoter of the invention operably linked to a nucleic acid encoding a sequence of interest, e.g., a FAR enzyme, can be cultured under a variety of conditions. A promoter of the invention is active in both “rich” medium and a medium that is a minimal media that lacks one or more amino acids. Thus, in one non-limiting example, a yeast host cell is cultured in a “rich medium” comprising complex sources of nitrogen, salts, and carbon. An example of such a medium is YP medium, which comprises yeast extract, peptone and glucose. In other non-limiting embodiments, the aqueous nutrient medium for growing a host cell comprising an expression cassette comprising a promoter of the invention operably linked to a polynucleotide encoding a protein of interest comprises a mixture of Yeast Nitrogen Base (Difco™) in combination supplemented with an appropriate mixture of amino acids, e.g., SC medium. In particular aspects of this embodiment, the amino acid mixture lacks one or more amino acids, thereby imposing selective pressure for maintenance of an expression vector within the recombinant host cell. In further embodiments, a media for cultivating yeast cells may be a nitrogen limitation medium that does not contain added nitrogen, e.g., a medium that contains glucose, e.g., about 16% glucose, potassium phosphate, thiamine, iron sulfate, magnesium sulfate, manganese sulfate and a buffers such as MES. An example of such a limitation medium contains 120 g/L glucose, 1 g/L potassium phosphate, 0.25 mg/L thiamine, 0.1 mg/L iron sulfate, 0.25 mg/L magnesium sulfate, 0.03 mg/L manganese sulfate, and 100 mM MES pH 5. In some embodiments, components such as magnesium and phosphate may be omitted.

In some embodiments, the yeast cell is cultured under conditions and for a suitable period of time to convert an assimilable carbon substrate to desired end products, e.g., fatty alcohols or fatty acyl-CoA derivatives. Carbon substrates are available in many forms and include renewable carbon sources and the cellulosic and starch feedstock substrates obtained therefrom. Exemplary carbon substrates, include, but are not limited to, monosaccharides, disaccharides, oligosaccharides, saturated and unsaturated fatty acids, succinate, acetate and mixtures thereof. Further carbon sources include, without limitation, glucose, galactose, sucrose, xylose, fructose, glycerol, arabinose, mannose, raffinose, lactose, maltose, and mixtures thereof. The culture media can include, e.g., feedstock from a cellulose-containing biomass, which in the context of the present invention, may also contain hemicellulose; a lignocellulosic biomass; or a sucrose-containing biomass.

In some embodiments, “fermentable sugars” are used as the carbon substrate. “Fermentable sugar” means simple sugars (monosaccharides, disaccharides, and short oligosaccharides) including, but not limited to, glucose, fructose, xylose, galactose, arabinose, mannose, and sucrose. In one embodiment, fermentation is carried out with a mixture of glucose and galactose as the carbon substrate. In another embodiment, fermentation is carried out with glucose alone to accumulate biomass. In still another embodiment, fermentation is carried out with a carbon substrate, e.g., raffinose, to accumulate biomass. In some embodiments, the carbon source is from cellulosic and starch feedstock derived from but not limited to, wood, wood pulp, paper pulp, grain, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass, and mixtures thereof.

In one embodiment, a method of making fatty acyl-CoA derivatives using an expression construct comprising a promoter of the invention operably linked to a polynucleotide encoding a FAR enzyme further includes the steps of contacting a cellulose-containing biomass with one or more cellulases to yield fermentable sugars, and contacting the fermentable sugars with a microbial organism as described herein. In one embodiment, the microbial organism is a yeast (e.g., Y. lipolytica) and the fermentable sugars comprise glucose, xylose, fructose and/or sucrose.

The recombinant microorganisms comprising a promoter of the invention can be grown under batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out a temperature of about 10° C. to about 60° C., about 15° C. to about 50° C., about 20° C. to about 45° C., about 20° C. to about 40° C., about 20° C. to about 35° C., or about 25° C. to about 45° C. In one embodiment, the fermentation is carried out at a temperature of about 28° C. and/or about 30° C. It will be understood that, in certain embodiments where thermostable host cells are used, fermentations may be carried out at higher temperatures.

In some embodiments, the fermentation is carried out for a time period of about 8 hours to 240 hours, about 8 hours to about 168 hours, about 8 hours to 144 hours, about 16 hours to about 120 hours, or about 24 hours to about 72 hours.

In some embodiments, the fermentation will be carried out at a pH of about 3 to about 8, about 4.5 to about 7.5, about 5 to about 7, or about 5.5 to about 6.5.

The following examples are offered to illustrate, but not to limit, the claimed invention.

### EXAMPLES

### Example 1

### FAR Expression Analysis

A set of promoters was chosen based on 1) predicted activity of genes in the glycolytic pathway; 2) expression in mid-exponential phase in rich media, as determined experimentally using DNA microarray analysis of global gene expression of Y. lipolytica strain DSMZ 1345; and 3) stable expression in early, mid, and late exponential phase in rich media, as determined by microarray analysis.

TABLE 1
Primers used to amplify promoters for restriction free cloning into FAR
expression constructs.: Legend: SEQ ID NOS: 44-109.

| Promoter | Primer | sequence |
|---|---|---|
| YALI0E12683g | E12683g_primerA | CTGCTGCTGGGTGGCCATGATTGATTAGTTTGGGTGTTGGT |
| | E12683g_primerB | GACCATGATTACGCCAAGCTTGGCAATTGGTGGGATCCTTTTC |
| | E12683g_primerC | CAGCGTTTAGGGGTTGCAAAGCCAATTGGTGGGATCCTTTTCA |
| YALI0F09185g | F09185g_primerA | CTGCTGCTGGGTGGCCATTGTAACTGTGGTGTGAATTTCTC |
| | F09185g_primerB | GACCATGATTACGCCAAGCTTGGGTCGGTATCTCGCTCAATGAC |
| | F09185g_primerC | CAGCGTTTAGGGGTTGCAAAGCGTCGGTATCTCGCTCAATGAC |
| YALI0B05610g | B05610g_primerA | CTGCTGCTGGGTGGCCATTTTCGTTTTGTTTGGTTGTGG |
| | B05610g_primerB | GACCATGATTACGCCAAGCTTGGGTTTGATGATGGCCCATGA |
| | B05610g_primerC | CAGCGTTTAGGGGTTGCAAAGCCATGACGAGCGAATTCTCAAG |
| YALI0D14850g | D14850g_primerA | CTGCTGCTGGGTGGCCATTGTCAGGTACTGTGTGGGGT |
| | D14850g_primerB | GACCATGATTACGCCAAGCTTGGAAGGAAAACCACGTCATTGTG |
| | D14850g_primerC | CAGCGTTTAGGGGTTGCAAAGCGAAAACCACGTCATTGTGTG |
| YALI0F24673g | F24673g_primerA | CTGCTGCTGGGTGGCCATTGTTGTGTTGGTATGGGTTGTG |
| | F24673g_primerB | GACCATGATTACGCCAAGCTTGGGACAGGTGGGTCGTCTTTTG |
| | F24673g_primerC | CAGCGTTTAGGGGTTGCAAAGCGACAGGTGGGTCGTCTTTTG |
| YALI0E34749g | E34749g_primerA | CTGCTGCTGGGTGGCCATGTTGTTTGTAGATGTTACTGTTCAATTG |
| | E34749g_primerB | GACCATGATTACGCCAAGCTTGGTGGACTCCATAACTTGACAAGAG |
| | E34749g_primerC | CAGCGTTTAGGGGTTGCAAAGCCATAACTTGACAAGAGGGACATTAATC |
| YALI0E01298g | E01298g_primerA | CTGCTGCTGGGTGGCCATTGTTGTTTTTGTGTAATGAATAAGAGATATTC |
| | E01298g_primerB | GACCATGATTACGCCAAGCTTGGGTTCGTACCAGCACCAATGTTAG |
| | E01298g_primerC | CAGCGTTTAGGGGTTGCAAAGCGTTCGTACCAGCACCAATGTTAG |
| YALI0E19206g | E19206g_primerA | CTGCTGCTGGGTGGCCATATGTTGTGTGTGTAGTGTTGTTGTG |
| | E19206g_primerB | GACCATGATTACGCCAAGCTTGGGACGTGGTACCGAGGCTG |
| | E19206g_primerC | CAGCGTTTAGGGGTTGCAAAGCGACGTGGTACCGAGGCTG |
| YALI0F07711g | F07711g_primerA | CTGCTGCTGGGTGGCCATTGTGTTTGTGTGTTGGTGTGTC |
| | F07711g_primerB | GACCATGATTACGCCAAGCTTGGACAAAAGGTAGCAGAAGTATACTGTAT |
| | | ACTCA |
| | F07711g_primerC | CAGCGTTTAGGGGTTGCAAAGCGTAGCAGAAGTATACTGTATACTCACTC |
| | | TTTC |
| YALI0D07634g | D07634g_primerA | CTGCTGCTGGGTGGCCATGTTCAATTGGTGTGTTTGGGT |
| | D07634g_primerB | GACCATGATTACGCCAAGCTTGGTGACCTCATAGAAACAAAGTTGACTG |
| | D07634g_primerC | CAGCGTTTAGGGGTTGCAAAGCGACCTCATAGAAACAAAGTTGACTGAC |
| YALI0B00792g | B00792g_primerA | CTGCTGCTGGGTGGCCATTGTGTGTTGAGATGTTGTGTGTG |
| | B00792g_primerB | GACCATGATTACGCCAAGCTTGGGTGTCATTTTCTAAGACATTTAGCGA |
| | B00792g_primerC | CAGCGTTTAGGGGTTGCAAAGCGTGTCATTTTCTAAGACATTTAGCGAG |
| YALI0G16819g | G16819g_primerA | CTGCTGCTGGGTGGCCATGGTGATAAATGTGTGGTTAGAC |
| | G16819g_primerB | GACCATGATTACGCCAAGCTTGGCATTAGCTAGCTAGAGTCCAGC |
| | G16819g_primerC | CAGCGTTAGGGGTTGCAAAGCCATTAGCTAGCTAGAGTCCAGCTTC |
| YALI0E18568g | E18568g_primerA | CTGCTGCTGGGTGGCCATTTTTGTGTGTCTTGGTTGGATG |
| | E18568g_primerB | GACCATGATTACGCCAAGCTTGGAGATGGTGCTGCCAGGAG |
| | E18568g_primerC | CAGCGTTTAGGGGTTGCAAAGCGATGGTGCTGCCAGGAG |
| YALI0F05214g | F05214g_primerA | CTGCTGCTGGGTGGCCATTTTGAATGTAGTTGTGTTGTATGTACGA |
| | F05214g_primerB | GACCATGATTACGCCAAGCTTGGTTTCGGCGTGCAAAATC |
| | F05214g_primerC | CAGCGTTTAGGGGTTGCAAAGCGTGCAAAATCGCACGAAC |
| YALI0D16357g | D16357g_primerA | CTGCTGCTGGGTGGCCATTGTCGGTGTTTTGAAGC |
| | D16357g_primerB | GACCATGATTACGCCAAGCTTGGCATTTATCGACCCATCGAC |
| | D16357g_primerC | CAGCGTTTAGGGGTTGCAAAGCGACCCTCCCCGACATGTC |
| YALI0D00627g | D00627g_primerA | CTGCTGCTGGGCCATAACGATTTAACTGGGTAAAATAATATG |
| | D00627g_primerB | GACCATGATTACGCCAAGCTTGGCACCGACACACGGAAAG |
| | D00627g_primerC | CAGCGTTTAGGGGTTGCAAAGCCATAGATGTTACTCATGCCATGGTAC |
| YALI0D14344g | D14344g_primerA | CTGCTGCTGGGTGGCCATTGTGGTGGTGGTGGTGGT |
| | D14344g_primerB | GACCATGATTACGCCAAGCTTGGACTCCTTCCAGAAAAATGTGATG |
| | D14344g_primerC | CAGCGTTTAGGGGTTGCAAAGCCTCCTTCCAGAAAAATGTGATG |
| YALI0B02178g | B02178g_primerA | CTGCTGCTGGGTGGCCATCGTTTTGAGAGTCTGGTGGAGT |
| | B02178g_primerB | GACCATGATTACGCCAAGCTTGGCTCGTCGTCGACCATCTCTC |
| | B02178g_primerC | CAGCGTTTAGGGGTTGCAAAGCCTCGTCGTCGACCATCTCTC |
| YALI0B18150g | B18150g_primerA | CTGCTGCTGGGTGGCCATCTGTGTTAGTTCGGTTTGATGTG |
| | B18150g_primerB | GACCATGATTACGCCAAGCTTGGGTTAGTGTACGTACCGAGGGTG |
| YALI0C11341g | C11341g_primerA | CTGCTGCTGGGTGGCCAATTGTGTTGTGTGTTCGAAATGTG |
| | C11341g_primerB | GACCATGATTACGCCAAGCTTGGGTATGCAGAGTGCACCCAATTAG |
| YALI0A21307g | A21307g_primerA | CTGCTGCTGGGTGGCCATTGAATGCCTGAGAGTGGGGT |
| | A21307g_primerB | GACCATGATTACGCCAAGCTTGGCAGGTCTGTGATTGGTTGAAAACTG |
| YALI0D01441g | D01441g_primerA | CTGCTGCTGGGTGGCCATTGTGGTGGTGTTGTGTGTG |
| | D01441g_primerB | GACCATGATTACGCCAAGCTTGGGATGGTTGCTCTCAAAGCTC |
| YALI0E25982g | E25982g_primerA | CTGCTGCTGGGTGGCCATAGTGCAGGAGTATTCTGGGGA |
| | E25982g_primerB | GACCATGATTACGCCAAGCTTGGCATACGGAGAAACCACAGTTTCA |
| YALI0B02332g | B02332g_primerA | CTGCTGCTGGGTGGCCATTGGGATATGGAGAGTTGAGTG |
| | B02332g_primerB | GACCATGATTACGCCAAGCTTGGCAACGTCAATTGAGGGTGT |

The promoters to be tested were isolated from Yarrowia lipolytica genomic DNA by PCR. The sequences of the primers used to produce promoters that were active in the assay described in the following paragraph are provided in Table 1. PCR was performed using the primers listed in Table 1 as “primer A” and “primer B”. Primers contained 5′ overhangs to allow for introduction of the amplified promoters immediately upstream of the M. algicola FAR gene in plasmid pCEN411 (U.S. Patent Application Publication No. 20110000125) by the method of restriction free cloning (van den Ent et al., J. Biochem. and Biophys. Methods 67: 67-74, 2006). The sequence of the codon-optimized FAR gene used for this analysis is provided in SEQ ID NO:40. The gene encodes a FAR protein of SEQ ID NO:37. In each case, a sequence of 1500 bp immediately upstream of the gene of interest was employed. For analysis of FAR protein expression levels, the resulting plasmids were transformed into Y. lipolytica strain CY-201 using routine transformation methods, see, e.g. Chen et al., Appl. Microbiol. Biotechnol. 48: 232-235, 1997. The promoter from the translation elongation factor-1a (TEF) gene from Yarrowia lipolytica (U.S. Pat. No. 6,265,185) (SEQ ID NO:41) was used as a control.

Strains harboring the FAR expression plasmids were grown to mid-exponential phase in YPD media (1% yeast extract, 2% peptone, and 8% glucose) supplemented with 500 μg/mL hygromycin. Cells were harvested by centrifugation and lysed by the sodium hydroxide/SDS method (Kushnirov V., “Rapid and reliable protein extraction from yeast” Yeast 16: 857-860, 2000). Cell lysates were separated by SDS-PAGE then transferred to nitrocellulose membranes for Western blotting with a polyclonal antibody raised against an immunogenic peptide from the FAR sequence (ERLRHDDNEAFETFLEER, SEQ ID NO:110). Blots were then probed with IRDye 800CW goat anti-rabbit antibody (Licor #926-32211), and FAR expression was quantitated using an Odyssey infrared imager (Licor). From this experiment, twenty-four promoters were identified as suitable for FAR expression (Table 2). The measured level of FAR protein in these twenty-four strains varied from 0.5× to 9× over the control strain expressing FAR from the TEF promoter.

TABLE 2
FAR expression level from different promoters
in exponential phase cultures

| | Fold imp. over |
|---|---|
| Promoter | pos. ctrl |
| YALI0E12683p | +++++ |
| YALI0F09185p | ++++ |
| YALI0B05610p | ++++ |
| YALI0D14850p | +++ |
| YALI0F24673p | +++ |
| YALI0E34749p | +++ |
| YALI0E01298p | ++ |
| YALI0E19206p | ++ |
| YALI0F07711p | ++ |
| YALI0D07634p | ++ |
| YALI0B00792p | ++ |
| YALI0F16819p | ++ |
| YALI0E18568p | ++ |
| YALI0F05214p | ++ |
| YALI0D16357p | ++ |
| YALI0D00627p | ++ |
| YALI0D14344p | ++ |
| YALI0B02178p | + |
| YALI0B18150p | + |
| YALI0C11341p | + |
| YALI0A21307p | + |
| YALI0D01441p | + |
| YALI0E25982p | + |
| YALI0B02332p | + |

+ = promoter activity from about 0.5X up to 1.00 relative to the TEF control promoter.
++ >1.0 fold improvement relative to the TEF control promoter
+++ = >2.0 fold improvement relative to the TEF control promoter
++++ = >3.0 fold improvement relative to the TEF control promoter
+++++ = >5.0 fold improvement relative to the TEF control promoter

Promoters that were active in YPD media were cloned by the restriction-free method into a plasmid harboring a DNA construct that enabled integration of a FAR expression cassette into a specific location in the Y. lipolytica genome. In this case, promoters were amplified using “primer A” and “primer C” listed in Table 1. The resulting integrating constructs contained a M. algicola FAR expression cassette (with the variable promoter) and a second expression cassette that encoded hygromycin resistance. The DNA encoding these expression cassettes was flanked on either side by ˜1 kb of Y. lipolytica DNA that acted to target this DNA to a specific intergenic site on chromosome E.

Integration constructs were amplified by PCR and transformed into Y. lipolytica strain CY-201. The resulting integrants were grown in YPD media then transferred to a nitrogen limitation medium (NLM) that included 120 g/L glucose, 1 g/L potassium phosphate, 0.25 mg/L thiamine, 0.1 mg/L iron sulfate, 0.25 mg/L magnesium sulfate, 0.03 mg/L manganese sulfate, and 100 mM MES pH 5 for analysis of fatty alcohol production. Fatty alcohol (FOH) titer was measured by GC-FID after 24 incubation in nitrogen limitation media. The fatty alcohol production obtained for various integrants is shown in Table 3. This identified promoters YAL0E12683p, YALI0E19206p, and YALI0E34749p as particularly effective for FAR expression in nitrogen limitation medium.

TABLE 3
Fatty alcohol production in nitrogen limitation medium
in integrants having promoter-FAR expression cassettes.

| Promoter | FOH, g/L |
|---|---|
| Neg. control | − |
| TEF promoter control | ++ |
| YALI0E12683p | +++ |
| YALI0E19206p | +++ |
| YALI0E34749p | + |
| YALI0F05214p | + |
| YALI0F09185p | − |
| YALI0B05610p | − |
| YALI0D14850p | − |
| YALI0F24673p | − |
| YALI0E01298p | − |
| YALI0F07711p | − |
| YALI0D07634p | − |
| YALI0B00792p | − |
| YALI0F16819p | − |
| YALI0E18568p | − |
| YALI0D16357p | − |
| YALI0D00627p | − |
| YALI0D14344p | − |
| YALI0B02178p | − |

“−” = level equivalent or similar to negative control CY-201
++ = positive control level
+++ = level above positive control
+ = level less than positive control

### Example 2

### Deletion Analysis of the YALI0E19206, YALI0E12683, and YALE34749 Promoters

To further evaluate the YALI0E19206, YALI0E12683, and YALE34749 promoters, a series of truncations were made in the pCEN411-derived plasmids containing the promoters (see, Example 1). In each case, 250 bp, 500 bp, 750 bp, 1000 bp, or 1250 bp were deleted from the 5′ end of the promoter using PCR to amplify the desired region of the plasmid. For each reaction, the common primer pCEN354-SDM-R, which anneals to the vector sequence immediately upstream of the primers, was combined with a second, unique primer (see Table 4 for primer sequences). PCR primers were phosphorylated at their 5′ ends to facilitate plasmid circularization by T4 DNA Ligase. Circular DNA was transformed into E. coli and then purified using standard DNA methods. The resulting promoter truncation plasmids were transformed into Y. lipolytica CY-201 using routine transformation methods (see, e.g. Chen et al., Appl. Microbiol. Biotechnol. 48: 232-235, 1997).

TABLE 4
Primers for promoter deletions.

| Primer name | DNA sequence |
|---|---|
| pCEN354-SDM-R | ccaagcttggcgtaatcatggtc |
| E19206p-1250bp | aacgccaacaggatccgattc |
| E19206p-1000bp | ttctcctccagtatcatttttctatccgt |
| E19206p-750bp | caatatcgacgcagatacacactctca |
| E19206p-500bp | acccgataatatcgtccatatggctc |
| E19206p-250bp | taaaccagttgcacacgtttccgt |
| E12683p-1250bp | gagggcggcgctataacgtagt |
| E12683p-1000bp | ttgagcacggactccaatatg |
| E12683p-750bp | gaagcgttgtttttggggcaag |
| E12683p-500bp | ggacaatgaatcgatggagacatg |
| E12683p-250bp | cagcgaatggcgtcctcca |
| E34749p-1250bp | agcaatcaaaatacttgcaaataccggtac |
| E34749p-1000bp | agtgttttttctatccaaaagggggcca |
| E34749p-750bp | tttcgtgatctcattcaatgatttctgtatg |
| E34749p-500bp | agtgacctctgtgggtctcttttttgt |
| E34749p-250bp | gaggggaattctacctttggattgtttc |

Legend: SEQ ID NOS: 111-126.

For analysis of FAR expression level, the transformed strains were grown to mid-exponential phase in YPD media (1% yeast extract, 2% peptone, and 8% glucose) supplemented with 500 μg/mL hygromycin. FAR protein expression level was analyzed as described in Example 1. Briefly, cell lysates were prepared by the sodium hydroxide/SDS method then separated by SDS-PAGE and transferred to nitrocellulose membrane. Blots were incubated with the anti-FAR polyclonal antibody, then probed with IRDye 800CW goat anti-rabbit antibody (Licor #926-32211). FAR expression was quantitated using an Odyssey infrared imager (Licor). Table 5 shows the activity of the promoter truncations relative to the 1500 bp promoter. For each of the three promoters, the truncated promoters retained the activity of the 1500 bp promoter.

TABLE 5

| | E34749 promoter | E12683 promoter | E19206 promoter |
|---|---|---|---|
| Promoter | (SEQ ID | (SEQ ID | (SEQ ID |
| size | NOS: 11-15) | NOS: 1-5) | NOS: 6-10) |
| 1500 bp | 100% | 100% | 100% |
| 1250 | Not evaluated | Not evaluated | + |
| 1000 bp | + | + | + |
| 750 bp | + | + | + |
| 500 bp | + | + | + |
| 250 bp | + | + | + |

+ = FAR expression similar to 1500 bp promoter

### Example 3

### Analysis of Variant Promoters

This example illustrates assessing activity of a variant promoter in a reporter expression system. SEQ ID NO:10 (YALI0E19206 promoter sequence) is cloned into a vector and variants are made by random mutagenesis methods known in the art. Several variants are generated. Two variant sequences (SEQ ID NO:42 and 43, with 95% and 92% identity, respectfully, to SEQ ID NO:10), are tested. The variant promoter sequence is cloned into an expression vector such that the variant sequence is upstream of a luciferase reporter gene sequence immediately before the ATG translation start site. The expression vector is introduced into Yarrowia lipolytica and luciferase activity is assessed in the yeast cells in comparison to the activity obtained with the wildtype type promoter SEQ ID NO:10. Promoter activity is then evaluated for the ability to drive expression of a FAR protein (SEQ ID NO:37). The variant promoter is cloned into an expression vector upstream of the FAR gene. The yeast strain is transformed with the expression construct. The transformed strain is grown to mid-exponential phase in YPD media (1% yeast extract, 2% peptone, and 8% glucose) supplemented with 500 mg/mL hygromycin. FAR protein expression level is analyzed by immunoassay using an anti-FAR polyclonal antibody and FAR expression is quantitated. Variant promoters for use in the invention preferably retain at least 90% of the activity of the wildtype promoter.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An expression construct comprising a promoter operably linked to a heterologous DNA sequence encoding a protein, wherein the promoter comprises:
(a) a nucleotide sequence having at least 80% sequence identity to nucleotides 1-100 of SEQ ID NO:15 or
(b) a nucleotide sequence having at least 80% sequence identity to nucleotides 1-100 of SEQ ID NO:10.

2. The expression construct of claim 1, wherein the promoter comprises a nucleotide sequence having at least 90% sequence identity to nucleotides 1-100 of SEQ ID NO:15 or nucleotides 1-100 of SEQ ID NO:10.

3. (canceled)

4. (canceled)

5. The expression construct of claim 1, wherein the promoter comprises a nucleotide sequence having at least 80% sequence identity to SEQ ID NO:15 or SEQ ID NO:10.

6. The expression construct of claim 5, wherein the promoter comprises a nucleotide sequence having at least 90% sequence identity to SEQ ID NO:15 or SEQ ID NO:10.

7. (canceled)

8. (canceled)

9. The expression construct of claims claim 1 that does not comprise nucleotides 1-100 of SEQ ID NO:11 or SEQ ID NO:6.

10.-18. (canceled)

19. The expression construct of claim 1, wherein the protein is an enzyme.

20. The expression construct of claim 19, wherein the enzyme is a reductase, a synthase, a dehydrogenase, an invertase, an esterase, or a cellulase.

21. The expression construct of claim 20, wherein the enzyme is a fatty acyl reductase (FAR).

22. An expression construct comprising a promoter operably linked to a heterologous DNA sequence encoding a fatty acyl reductase (FAR) protein, wherein the promoter comprises a nucleotide sequence having at least 80% sequence identity to nucleotides 1-100 of SEQ ID NO:5.

23. The expression construct of claim 22, wherein the promoter comprises a nucleotide sequence having at least 90% sequence identity to nucleotides 1-100 of SEQ ID NO:5.

24. (canceled)

25. (canceled)

26. The expression construction of claim 22, wherein the promoter comprises a nucleotide sequence having at least 80% sequence identity to SEQ ID NO:5.

27. The expression construct of claim 26, wherein the promoter comprises a nucleotide sequence having at least 90% sequence identity to SEQ ID NO:5.

28.-51. (canceled)

52. The expression construct of claim 21, wherein the FAR is from a Marinobacter species, or is a variant thereof.

53. The expression construct of claim 52, wherein the FAR is recombinant.

54. A host cell comprising the expression construct of claim 1.

55. The host cell of claim 54, wherein the expression construct is integrated into a chromosome of the host cell.

56. The host cell of claim 54, wherein the host cell is a yeast.

57. The host cell of claim 56, wherein the host cell is an oleaginous yeast.

58. The host cell of claim 57, wherein the host cell is Yarrowia lipolytica.

59. A method for producing a protein in a host cell, comprising culturing a host cell of claim 54 under conditions in which the protein is produced in the cell.

60. An isolated nucleic acid having promoter activity, wherein the nucleic acid comprises:
(a) a nucleotide sequence having at least 80% sequence identity to nucleotides 1-100 of SEQ ID NO:15 or
(b) a nucleotide sequence having at least 80% sequence identity to nucleotides 1-100 of SEQ ID NO:10.

61.-70. (canceled)
